(19) United States
(12) Patent Application Publication
(10) Pub. No.: US 2025/0125056 A1
(43) Pub. Date: Apr. 17, 2025

(54) HEALTH INFORMATION PROCESSING SYSTEM

(71) Applicant: FORTIFIED HEALTH INC., Gainesville, VA (US)

(72) Inventor: Britt Massei, Gainesville, VA (US)

(21) Appl. No.: 18/688,539

(22) PCT Filed: Sep. 1, 2022
(86) PCT No.: PCT/US2022/075856

§ 371 Date: Mar. 1, 2024

Related U.S. Application Data
(60) Provisional application No. 63/239,761, filed on Sep. 1, 2021.

(51) Int. Cl. G16H 50/30; G16H 10/60; G16H 20/10

(52) U.S. Cl. CPC G16H 50/30; G16H 10/60; G16H 20/10

(57) ABSTRACT

Methods, systems, and apparatus, including computer programs encoded on a computer storage medium, for health information process. In one aspect, a system enables the collection of user specific health data from multiple different sources. —Account logins to multiple different sources are managed by the system. Data are cleaned in an intelligent and efficient manner. Sharing levels for different portions of data are set and the portions of data can be shared according to the share levels. Users can be queried when health metric values deviate from a baseline, and the responses can be used to determine potential causes of the deviations.

### CROSS-REFERENCE TO RELATED APPLICATIONS

This application is the benefit of and priority to U.S. Provisional Application No. 63/239,761, filed on Sep. 1, 2021, the contents of which are hereby incorporated by reference.

### BACKGROUND

This specification relates to health data processing systems.

Currently health data are fractured and siloed. There is data in a variety of systems (e.g., multiple hospital health systems, multiple user applications, multiple insurance companies, etc.) but the data are not linked or managed in a way to make efficient use of that data.

When presented with new tools to collect more meaningful data, consumers often decline due to the lack of meaningfulness of the data. The health industry claims to break down silos, all the while new ones are being built even faster. There is currently no cohesive, robust, home for health data from the perspective of the individual that provides the individual control over their data, the ability to share their data, and the ability to receive the insights and intelligence that can result from the managing of this data from an information building framework, rather than a data repository framework.

### SUMMARY

In general, one innovative aspect of the subject matter described in this specification can be embodied in methods that include the actions of: for each of a plurality of users: establishing, for the user, a unique account for the user; receiving, for the user, a set of data sources for the user, each data source being different from each other data source, and each data source requiring login credentials for the user; for at least one or more of the data sources, establishing, by the system and without user input, login credentials for the user; and; storing in association with the unique account for the user, the login credentials for the user; for the at least one or more data sources of the set of data sources for the user:

establishing, using the login credential for the data source established by the system, a connection to the data source, receiving, from the data source, health data specific to the user; and storing, in the unique account of the user, the health data for the user received from each data source in a data store. Other embodiments of this aspect include corresponding systems, apparatus, and computer programs, configured to perform the actions of the methods, encoded on computer storage devices.

In another aspect, the operations further comprise, for each unique account: for data sources from which health data is received, determining whether the health data received from the data source requires data cleaning; for a first data source for which health data is determined to not require data cleaning, storing the health data in the unique account of the user; for a second data source for which health data is determined to require data cleaning: determining whether changes resulting from the data cleaning trigger a user alert, in response to determining the changes resulting from the data cleaning trigger the user alert: notifying the user to validate the changes before persisting the changes, persisting the changes only in response to the user validating the change; and in response to determining the changes resulting from the data cleaning do not trigger the user alert, persisting the changes.

In another aspect, the operations further comprise, for each unique account: for each data source from which health data is received: determining whether the health data received from the data source requires data cleaning, the determining comprising: determining, for the health data received from the data source, a health data type; determining, for the health data type, health data values required for the health data type; determining whether the health data values in the health data received form the data source do not match the health data values required for the health data type; for each data source for which the health data values of the health data received does not match the health data values required for the health data type, determining the health data received from the data source requires cleaning; and for each data source for which the health data values of the health data received do match the health data values required for the health data type, determining the health data received from the data source does not require cleaning.

In another aspect, the operations further comprise, for health data from a data source determined to require cleaning: determining health data values that are missing from the health data; generating a query for the data source requesting the health data values that are determined to be missing from the health data; and sending the query to the data source.

In another aspect, the operations further comprise, for health data from a data source determined to require cleaning: determining health data values that are missing from the health data; determining that at least one of the health data values is an index value that references the health data values that are missing from the health data in a health data database; and querying the health data database using the index value to obtain the health data values that are missing from the health data; and augmenting the health data with the health data values obtained from the health data.

In another aspect, the operation of storing, in the unique account of the user, the health data for the user received from each data source, comprises: for at least one data source, receiving health data in a non-standardized format, the non-standardized format being a format different from a standardized format in which health data is stored in the data store; determining a conversion of the health data in the non-standardized format to the standardized format based on the non-standardized format; converting, based on conversation, the health data from the non-standardized format to the standardized format; and storing the converted health data in the standardized format in the data store.

In another aspect, the operations further comprise for each unique account: receiving, from the user of the account, share data specifying a plurality of share levels for the health data, wherein different portions of the health data are each associated with a different share level; and for each portion of the health data associated with a particular share level, enabling sharing of the health data according to the share level.

In another aspect, the operations further comprise, for a unique account of a user, receiving data describing a user obligation to share particular health data of the user with a third party; determining that the share level of the particular health data precludes sharing of the particular health data with the third party; in response to determining that the share level of the particular health data precludes sharing of the particular health data with the third party, determining a user preference for a share conflict; and performing a share resolution process according to the user preference.

In another aspect, determining a user preference for a share conflict comprises determining whether the user preference is an automatic override of the share level or a user sharing conformation; in response to determining the user preference is an automatic override of the share level, allowing, without user confirmation, sharing of the particular health data with only the third party; and in response to determining the user preference is user sharing confirmation, requesting the user confirm sharing of the particular health data with the third party.

In another aspect, the operations further comprise, for each unique account: determining, based on the health data of the user, that a health metric value has deviated from a baseline value; determining, based on the health metric value that has been determined to deviate from the baseline value, one or more questions to present to the user; presenting the one or more questions to the user and storing the responses from the user to the one or more questions; and based on the health metric values and the responses, determining potential causes that caused the health metric value to deviate from the baseline value.

In another aspect, the operations further comprise, for each unique account: determining, based on the health data of the user, that a health metric value triggers a health inquiry; determining, based on the health inquiry determination, one or more questions to present to the user; presenting the one or more questions to the user and storing the responses from the user to the one or more questions; and based on the health metric values and the responses, determining potential causes that caused the health metric value to trigger a health inquiry.

In another aspect, the operations further comprise, for each user: determining, from the health data stored for the user account of the user, health preferences; determining, from the health preferences, a set of type weights, wherein each type weight describes an estimated level of user interest in a particular heath service type; selecting, based on the type weights, one or more health service recommendations for the user; and providing, to the user, the one or more selected health service recommendations.

In another aspect, the one or more health service recommendations include adjusting share levels of health data, a health product recommendation, a health action recommendation, and a health content stream recommendation.

In another aspect, the operations further comprise: receiving, from one of the data sources, a health history request for a user, the health history request specifying a plurality of health data values of the user to be provided; in response to the health history request, providing, to the one of the data sources, the health data values of the user.

In another aspect, in response to the health history request, providing, to the one of the data sources, the health data values of the user, comprises: sending, to the user, a request for confirmation to share the health data values to the one of the data sources; and providing, in response to the health history request, to the one of the data sources, the health data values of the user only in response to receiving a confirmation from the user.

In another aspect, the health data for one or more users include, for each of the one or more users, medications the user is taking and a schedule for each medication, and the operations further comprising, for each user of the one or more users: determining a periodic dosing schedule for each medication, the dosing schedule indicating, for each medication, a time to administer the medication, the determining comprising: for each medication, accessing medication data describing dosing requirements and constraints, medication interactions with other medications, and side effects of the medications; determining, from the health data of the user, an activity pattern data of the user, the activity pattern data specifying user activities over multiple periodic dosing periods; based on the medication data and the activity pattern data of the user, determining a periodic dosing schedule that specifies, for a time period and for each medication, a time to administer the medication; sending, to a user device of a user for display to the user, the periodic dosing schedule; and monitoring the dosing schedule for the user, the monitoring comprising at each time to administer a medication, sending, to the user device, a notification to the user to administer the medication.

In another aspect, sending, to the user device, a notification to the user to administer the medication comprises sending, to an electronic pill package that stores the medications for one or more periodic dosing schedules, data that causes electronic pill package to generate an audible and/or visual notification for the user.

In another aspect, the audible or visual notification specifies a specific compartment of the electronic pill package that stores a particular medication to be administered.

In another aspect, based on the medication data and the activity pattern data of the user, determining a periodic dosing schedule that specifies, for a time period and for each medication, a time to administer the medication comprises determining the periodic dosing schedule for at least one medication based on a dependency of food ingestion.

In another aspect, the operations further comprise determining, based on the dosing schedule for a user, a quantity of medications to store in each of a plurality of pill receptacles in the electronic pill package; and providing data that describes, to the user, the quantity of medications to store in each of a plurality of pill receptacles in the electronic pill package.

In another aspect, the operations further comprise determining, for a user device, a set of goals for tracking, wherein the goals are specific to a particular condition; determining, based on health data of a user of a user device, a corresponding set of goal values for the set of goals; receiving, from one or more user devices of the user, data indicating progress of the user in achieving each goal value for each goal in the set of goals; determining, from the data indicating progress of the user in achieving each goal value for each goal in the set of goals, an overall goal achievement of the user; and providing, to the user device, data that causes the user device to display, for each goal in the set of goals, a progress measure in achieving the goal, and the overall goal achievement of the user.

In another aspect, the operations further comprise determining, for each goal of the set of goals for tracking, whether the goal is trackable by a user instrumentation; for each goal that is determined to be trackable by a user instrumentation: determining whether the user account receives data from the user instrumentation for the goal; and for each goal for which the user account does not data from the user instrumentation for the goal, generating data that causes a user device of the user to display a recommendation for one or more devices that perform the function of the user instrumentation; for each goal that is determined to not be trackable by a user instrumentation, periodically providing data to a user device of the user that causes the user device to display an input user interface in which the user may enter the data indicating progress of the user in achieving the goal value, and receiving, from the user device, the data indicating progress of the user in achieving the goal value.

In another aspect, the operations further comprise determining, based on health data of the user of the user device, for a particular goal of the set of goals, that the user cannot perform activities to progress in achieving the goal value, and in response: suspending the collection of data indicating progress of the user in achieving the particular goal value; and adjusting the determining of the overall goal achievement of the user by omitting data indicating progress of the user in achieving each goal value of the particular goal from the determination.

In another aspect, the operations further comprise determining, based on health data of the user of the user device, for a particular goal for which it was determined that the user cannot perform activities to progress in achieving the goal value, that the user can perform activities to progress in achieving the goal value, and in response: resuming the collection of data indicating progress of the user in achieving the particular goal value; and adjusting the determining of the overall goal achievement of the user by including the data indicating progress of the user in achieving each goal value of the particular goal from the determination.

Particular embodiments of the subject matter described in this specification can be implemented so as to realize one or more of the following advantages. The login credential storage and security management ties access to multiple health data accounts together, which enables access to disparate data sets from the health information processing system. This allows near seamless access for a user to the user's data records from multiple different sources but within a single system.

The health information processing system also processes the data records received from the disparate data sources that each may store data in different unstandardized formats, e.g., each format different from the formats of the health information processing system, and standardizes the storage format in the health information processing system. This facilitates further processing of the records by the health information processing system, such as machine learning, curation, and summarization without requiring the additional overhead of converting records had the records only been stored in the disparate data sources.

Data cleaning/laundry functions are performed by the health information processing system to ensure that data are complete, and/or that data that the user does not want stored in the health information processing system is removed. Such changes may be further subject to user confirmation. This allows for flexible control for a user over the user's data. When additional data are needed from particular data sources, the health information processing system may access the user's login credentials to access the data without user input, which facilitates up-to-date records curation.

The user may enable sharing preferences for each data to allow certain data to be shared with third parties. The health information processing system can monitor sharing preferences of a user and may determine when the sharing preferences are inconsistent with a user commitment. This ensures that user commitments to third parties are consistent with their sharing preferences without the user being required to manually check those preferences.

The health information processing system may process medicant schedule data, which may be one or more dosing schedules for prescription medications, supplement medicants, over the counter medicants, or any other medicants and determine, based on the interaction data and dosing requirements, an optimal dosing schedule. The schedule may be presented to the user, and/or may be provided to a medical device (such as an electronic pillbox), which causes the electronic device to provide audible and/or visual signals that indicate when certain mendicants are to be taken. The medical device may also include monitoring devices to determine whether the pillboxes are loaded with medicants correctly, and whether the medicants have been taken at the correct time, and notify the user when medicants are not taken correctly or at the correct time. This results in a practical application of an optimized dosing schedule within a particular and special purpose machine.

The illness metering system identifies particular recommendations for which no automated data recording device is available, and in response generates data recordation queries to the user and delivers the queries to the user by electronic means. The queries include response options or fields that allow the user to quickly and easily provide data for recordation and goal tracking. Accordingly, data recordation and data integrity is increased relative to systems in which the user must initiate manual data recordation.

The advantages above are not exhaustive and other advantages, technological improvements, and benefits can also be realized.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a system diagram of health information processing system 100.

FIG. 2 is a system diagram 200 of example data sources that provide data for a health processing system 100 member account 110.

FIG. 3 is a system flow diagram of a data laundry process 300.

FIG. 4 is a system flow diagram of a data collection process 400.

FIG. 5 is a system diagram of a data control process 500.

FIG. 6 is a diagram 600 of system interoperability.

FIG. 7 is a system flow diagram 700 of credential processing.

FIG. 8 is a system flow diagram 800 of user experience customization.

FIG. 9 is a system flow diagram 900 of data organization.

FIG. 10 is a system flow diagram 1000 for global sharing preferences.

FIGS. 11 and 12 is a system flow diagram 1100 and 1200 of adjusting sharing preferences.

FIG. 13 is a system flow diagram 1300 of a data compilation process in story form.

FIG. 14 is a system flow diagram 1400 of timeline processing.

FIG. 15 is a system flow diagram 1500 of health history processing.

FIG. 16 is a system flow diagram 1600 of processing data against baselines.

FIG. 17 is a system flow diagram 1700 of processing multiple inputs for a comprehensive analysis.

FIG. 18 is a system flow diagram 1800 of detailed tracking prescriptions and supplements.

FIG. 19 is a system flow diagram 1900 for pill package medical device processing.

FIG. 20 is a system flow diagram 2000 for processing user-provided records.

FIG. 21 is a system flow diagram 2100 of data collection and processing.

FIG. 22 is a system flow diagram 2200 of a question generation process for data collection.

FIG. 23 is a system flow diagram 2300 of a data insight process.

FIG. 24 is a system flow diagram 2400 of a single variable insight process.

FIG. 25 is a system flow diagram 2500 of a multi-variable insight process.

FIG. 26 is a system flow diagram 2600 of another multi-variable insight process.

FIG. 27 is a system flow diagram 2700 of a financial distillery process.

FIG. 28 is a system flow diagram 2800 of a legal distillery process.

FIG. 29 is a system flow diagram 2900 of another query process.

FIG. 30 is a system flow diagram 3000 of a content curation process.

FIG. 31 is a system flow diagram 3100 of a health marketplace process.

FIG. 32 is a system diagram 3200 of a Health Care Provider (HCP) product suite system.

FIG. 33 is a system flow diagram 3300 of an HCP record management process.

FIG. 34 is a system flow diagram 3400 of an HCP Electronic Health Records (HER) process.

FIG. 35 is a system flow diagram 3500 of interactions between an HCP and member account.

FIG. 36 is a system flow diagram 3700 of beneficiary proposal process.

FIG. 37 is a system flow diagram 3700 of illness metering process.

FIG. 38 is a system flow diagram 3800 of tracking lifestyle goals generated from the process of FIG. 37.

FIG. 39 is an illustration of a user interface 3900 of an illness metering application.

Like reference numbers and designations in the various drawings indicate like elements.

### DETAILED DESCRIPTION

### Overview

The systems and methods described herein provide an interoperable platform that not only pulls members' (also referred to as “users”) data from countless sources but provides a framework for communicating with and among the system and third-party data sources and tools, fills many voids with system input tools, and converts the amassed data into valuable insights and actionable information powered by novel system tools as well as interoperable third-party tools.

In some implementations, health content from around the globe, ranging from legacy to cutting edge, traditional and nontraditional, are collected and converted to personalized information, based on the member's stored data and on their preferences for data sources and type. Rather than sift through countless publications, emails, social media feeds and more, searching for the latest news on their personal health needs, members' access relevant, highly personalized, distilled content, linked to insight, action and alert tools.

In some implementations, a marketplace aggregates all things health, from apps and devices to books and subscriptions, to health care providers and insurers. The massive barrage of data is not apparent to the member, as the system delivers succinct, personalized information and recommendations. Through the utilization of the wide breadth of data stored in the system, combined with member inputs, a novel ratings system will arm members to efficiently find and purchase the best products and services to suit their needs.

In some implementations, interested members are provided the means to share their data with beneficiaries including both trusted individuals, e.g., loved ones, care takers, health care providers (HCPs), etc., and third party organizations, e.g., those conducting scientific or market research, product and service providers, payers, government, etc.

HCPs enjoy benefits beyond the simple ability to view member-shared data, through an innovative on-platform electronic health record (EHR) system, fully integrated in real-time with the member's data and benefitting from the ML/A-driven insight and action tools. This enables HCPs to focus their efforts on practicing medicine by reducing administrative burdens.

In some implementations, members can opt to share their data with beneficiaries. The system's structured share programs mandate organization communication and transparency with the members, while encouraging member participation and even member-initiated research. This enables opportunities for researchers, from those conducting foundational research to those in clinical research, to instantly have access to millions of targeted individuals, who can share already-collected, longitudinal personal health data at the click of a button. Additionally, the system provides researchers with on-platform tools utilizing machine learning and artificial intelligence, to make the most efficient and effective use of the data.

As described above, many of the processes described in the system flows are implemented by machine learning and artificial intelligence. However, in variations of the HIPS 100, other decision making, automated processes can be used, such as predefined ruleset processes that are not machine learned, heuristics, and any other appropriate machine based process that can take one or more inputs for processing and predict, identify or estimate one or more outputs based on the inputs. Thus, while many of the processes are described in the context of machine learning and artificial intelligence, non-ML/AI processes are within the scope of the disclosure.

These features and additional features are described in more detail below.

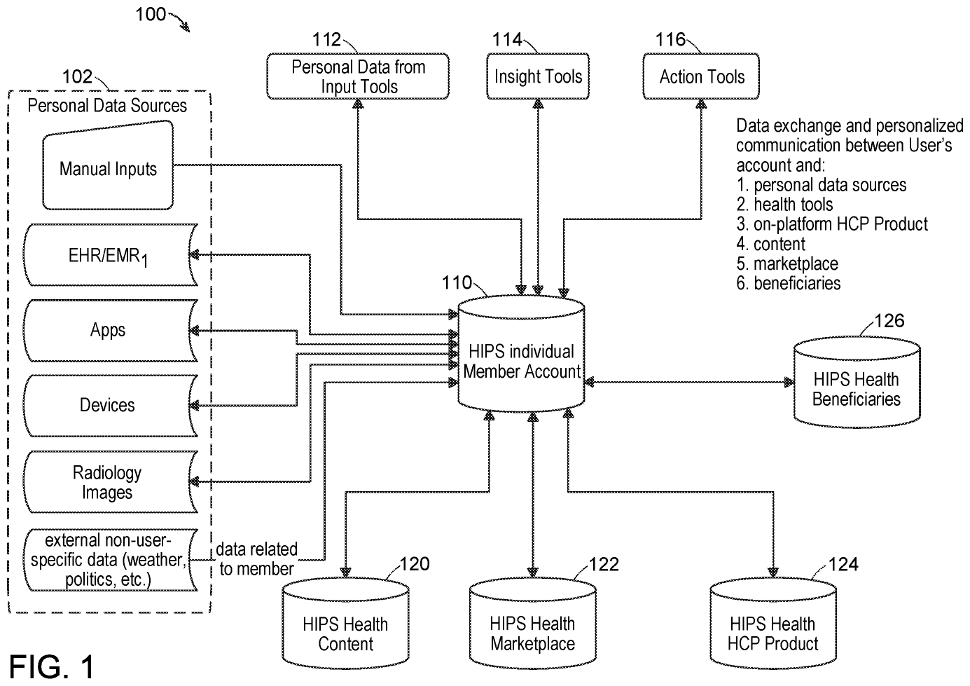

FIG. 1 is a system diagram of health information processing system 100. The system is implemented in one or more computers in a computer network, such as a local area network (LAN), wide area network (WAN), the Internet, or a combination thereof. Likewise, as described below, the devices of Health (Tare Providers (HCPs) and users/members are also implemented in one or more computer devices. For ease of illustration and to avoid clutter in the drawings, the diagrammatic representations of these devices are omitted in the system and flow diagrams.

The health information processing system 100 (HPS) empowers people to live better lives through better health by providing the framework and core components for a new ecosystem in health. HIPS provides a secure place for individual users (also referred to as “members”) to securely store all their health-related data 102 from different data sources, whether it is currently in an EHR/EMR/patient portal, collected within an app or via a device, stored on paper, CDs, or other computer files, residing solely in their memory, or data 112 collected through the use of input tools 221. For each member account 110, external, non-health specific data (e.g., local weather, etc.) can also be collected. The disparate data are pulled together into the HIPS individual member account 110, and the users may provide, by means of sharing preferences, the ability to share the data with third parties. Additionally, insight tools 114 and action tools 116 are also realized.

As will be described in more detail below, the HIPS 100 provides an interoperable platform that pulls a member's data from multiple sources and provides a framework for communicating with and among the HIPS and third-party data sources and tools, and converts the amassed data into standardized formats. The data are then processed to provide insights and actionable information powered by HIPS tools and, optionally, interoperable third-party tools.

The HIPS content 120 provides personalized content that is relevant to the member and is presented in distilled formats. For example, content may be provided with comparisons, charts, ratings, etc. The user can drill-down and discover why the content was chosen, which data in HIPS 100 contributed to the recommendation, etc.

In some implementations, a HIPS 100 health marketplace 122 aggregates the health data and additional health information provided from apps, devices, data from health care providers and insurers, and other data sources. By utilizing the data stored throughout HIPS 100, combined with member inputs, the system can implement a ratings system that will help members find and purchase the particular products and services to suit their needs.

The HCP product 124 is a suite of services and data that assist HCPs in providing care to the users that are patients of the HCPs and members of the HIPS 100. Finally, data for beneficiaries 126, which are individuals or organizations that will potentially benefit from receiving the shared data of users, is also stored in the HIPS 100 to facilitate discovery and partnership among beneficiaries and members.

### Individual Member Accounts

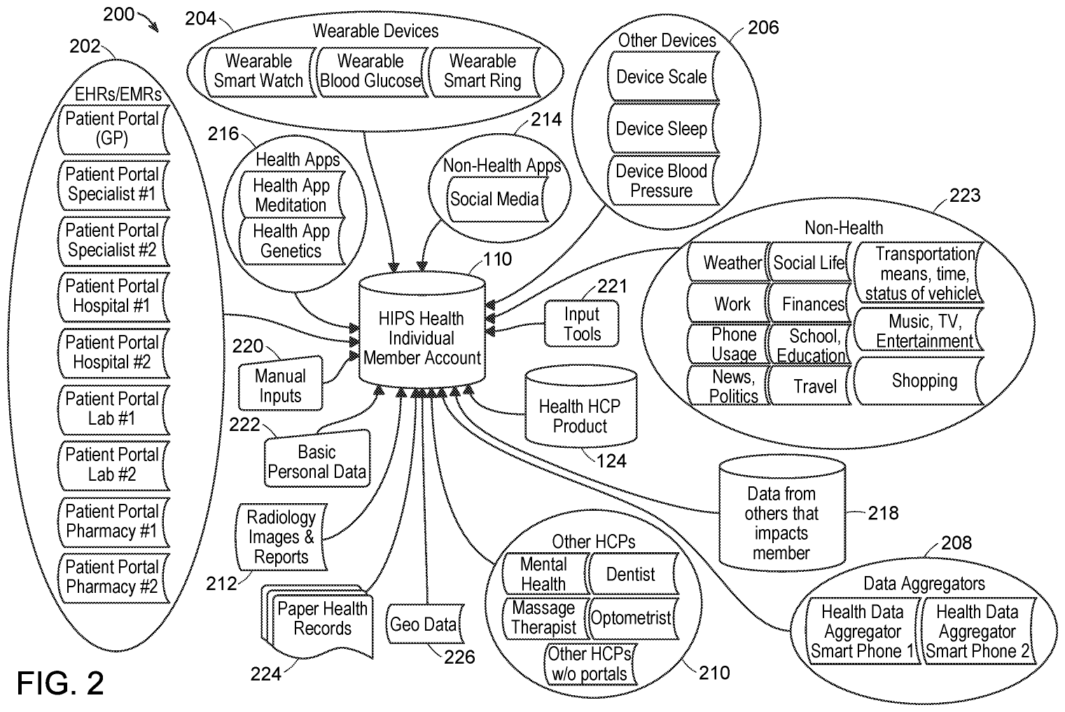

FIG. 2 is a system diagram 200 of example data sources that provide data for the health processing system 100 member account 110. The member account 110 stores data received and, optionally, standardized, from multiple difference sources, and may be accessed and used by multiple different tools. The HIPS 100 data is secured so that members maintain ownership, control, and accessibility of all their health-related data.

As illustrated in FIG. 2, the HIPS 100 receives data from multiple different sources. Each of these sources may store and/or provide data in different formats. In some implementations, the HIPS 100 stored data received from the data sources in a different format from the data formats stored in each data source. Thus, the HIPS 100 stores data in a standardized data format relative to the data formats of each data source. In particular, the HIPS 100, for one or more of the data sources, receives the health data in a non-standardized format that is different from a standardized format in which health data is stored in a data store of the HIPS 100.

The HIPS 100 then determines a conversion of the health data in the non-standardized format to the standardized format based on the non-standardized format. This conversion can be learned, for example, or can be done according to a predefined rule set. In the case of learning the conversion, in some implementations the HIPS 100 scans the records for key fields, and if key fields are found, determines a parameter type. The parameter types are then associated with the corresponding values, and the records are imported into the data schema that is used as the standard for the HIPS 100. Error processing can be implemented when unmatched data fields are found or expected data fields are missing. For example, an administrator can be notified for conversion resolution.

In the case of the latter, the administrator can create a rule set for records from each data source, and the records are imported according to the rule set.

Once the conversion is determined, the HIPS 100 converts, based on the determined conversion, the health data from the non-standardized format to the standardized format. The converted health data is then stored in the standardized format in the data store.

There are, in some implementations, different ways of collecting data. For example, collection types may be passive or active. Passive data collection does not require continual, active input from members; rather data are collected without continual intervention. Many devices, including smart watches and smart phones, automatically collect activity data like steps per day, heart rate, and more. This information can be automatically provided to the HIPS 100 system by means of a monitoring application or other data collection techniques, such as device, environmental data collection, ambient home health and wellness monitoring platforms, etc. Active data collection is a type of data collection that requires regular input and/or intervention, such as manually entering personal health history and daily food intake and manually entering exercise data.

There are also multiple data source types. These include medical data and clinical data, most of which are currently stored in EHRs, and also paper records and/or scanned paper records 224. Data sources also include non-medical Health data that includes data that was not created by or for a medical professional, but is health data such as that from tests, devices and apps, that are by design health data. Examples are weight, blood pressure monitoring, food sensitivity tests, and genetic tests. Other data includes “life data” such as data defining relationships, exercise, and diet, with data coming from a variety of manual inputs 220. External non-health data 223, such as weather, politics and societal events can also be accounted for, as such data may be indicative of certain health risks and/or benefits.

For example, a data source may include EHRs/portals 202. Complete records or even partial records may be provided, e.g., often only partial records are displayed in portals versus the complete records accessible by HCPs in the EHR. Examples of data are clinical reports and measurements, radiology reports and images 212, blood tests, electrocardiogram (EKG), and pill camera used for virtual endoscopies.

Fast Healthcare Interoperability Resources (FHIR) may resolve some issues with data importation. However, for EHRs that are not fully FHIR compliant, the member can provide HCP a code or ID for the HIPS 100 that is unique and secure for the member account, so that the HCP can send data, e.g., images, etc. directly to the HIPS member account. Other H-HCPs 210, such as dentists, therapists, etc. can also provide data to the HIPS 100.

Wearable devices 204 are another data source and facilitate passive and/or active detection. Wearables include smart phones, smart watches and blood glucose monitors or other wearable health monitoring devices. Wearables may also include embedded or subcutaneous devices, such as pacemakers, etc. Other devices 206 that collect health data, such as blood pressure meters, scales, etc. can also be used.

Application data can include health-related applications 216, such as dieting apps, exercise apps, etc., and non-health related applications 214, such as social media apps, reading apps, etc.

Data aggregators 208 can include data from health apps, DNA analysis companies, and the like. For example, a data aggregator can be a company that collects a single health data type, e.g., data from a step counter, or a company that collects multiple data types, e.g., steps, sleep hours, blood pressure, etc. Data aggregators can also include HCPs or any other entity that collects and aggregates health data for users.

Data 218 from others that impact the member may also be included. For example, illnesses or other life events of other members that are related to the member may be accounted for.

A variety of input tools 221 may also be available to the user for inputting basic personal data 222, health data, and other data. Such tools may be a web portal, an application, or a combination of both. Input tools are described in more detail below.

Still other sources can also be used to collect data. These include informational documents that are provided to individuals about procedures, conditions, vaccinations, etc. that may include symptoms to track. These data are not necessarily member data but are intended to improve the education and health of an individual and can be integrated into the member's record.

In some implementations, geotracking data 226 are recorded by using member's data collected on a smart phone, smart watch, or other geo-tracking device, subject to the user opting-in to allow such tracking. Activities and travel may be recorded.

In further implementations, the HIPS 100 may include shopping information based on the member identifier being linked to retailers and merchants. Again, the user may opt-in to this feature. Examples of shopping sites include online shopping and in-person like grocery stores. The HIPS 100 utilizes known purchases to guide the member for such inputs as foods consumed. HIPS can also guide purchases based on items that may be running low based on member consumption inputs.

In some implementations, indirect, globally stored datasets are incorporated into each member's records, utilizing links to personal data for which the member has opted-in. This data can include weather data, including allergens, humidity, temperatures, and more, based on the geotracking data. Events in the news, including pandemics, other types of health events, political news, etc., which all contribute to physical health and mental health, can also be incorporated into each member's records. Members may record their political or other group affiliation, within their HIPS record, so that relevant stories to that individual can properly be linked, correlated, and have insights drawn from. Further details about extent of involvement or personal impact, may be entered by the member to further strengthen the relevant correlations.

Members may also provide video to the HIPS 100. The member may record via video (or just audio), their experience, symptoms, etc. Video not only captures the words spoken and relevant images of the user and their inputs being discussed but shows measurable body language and speech that can be evaluated via AI programs for static and longitudinal changes to help detect conditions, e.g., Parkinson's disease.

In some implementations, the HIPS 100 provides the ability for the user to generate customized data records to record all data that does not yet have an app or a specifically designated place in HIPS 100. This enables collection of data a member wants to record, in any form. Members may then manually record the data, or, alliteratively, grant permission to apps (or app developers) to view the identified data and/or the type of data from those custom fields, to record or otherwise drive development of apps that will benefit the member in future data collection of that data.

### Data Curation/Cleaning/Laundry

The HIPS 100, in some implementations, conducts a data cleaning process. This process detects and corrects, or removes, corrupt or inaccurate records from a record set, table, or database and refers to identifying incomplete, incorrect, inaccurate or irrelevant parts of the data and then replacing, modifying, or deleting the dirty or coarse data. Data cleansing may be performed interactively with data conversion and mapping tools, or as batch processing through scripting.

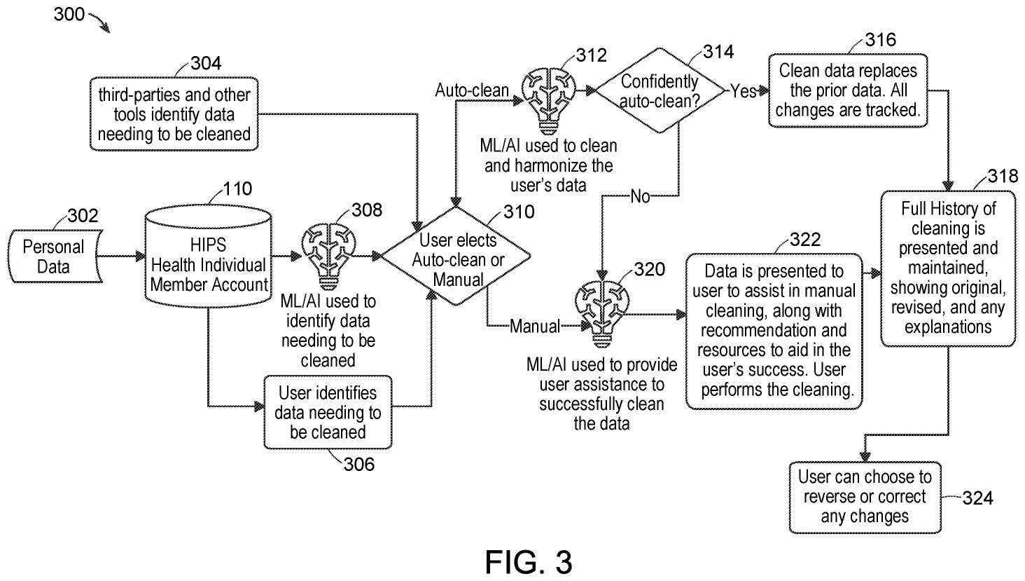

FIG. 3 is a system flow diagram 300 of a data laundry process. The data cleaning ensures the usefulness of the data. Wide-scale efforts are made to clean, harmonize, and validate the data in the HIPS 100. In operation, the members have an environment that displays data in need of member review and gives them the ability to clean the data by following the prompts provided. The cleaning can be done manually, automatically, or by a combination of both. For automated data cleaning, the HIPS 100 allows members to set alert levels for reviews to review all changes, changes at a specified level, or none. Review levels include a clear rating system that conveys the significance of each cleaning change as well as the confidence of each operation. All changes to data are tracked and can be readily accessed by the member and with the member's approval, by others.

In some implementations, the HIPS 100, for each unique member account, determines for data sources from which health data is received, whether the health data received from the data source requires data cleaning. For a first data source for which health data is determined to not require data cleaning, the HIPS 100 stores the health data in the unique account of the user. However, for a second data source for which health data is determined to require data cleaning, the HIPS 100 performs a cleaning process.

During the cleaning process, the HIPS 100 determines whether changes resulting from the data cleaning trigger a user alert. If the user alert is trigger, the HIPS 100 notifies the user to validate the changes before persisting the changes, and persists the changes only in response to the user validating the change. If a user alert is not triggered, then the changes are persisted.

The HIPS 100 can also determine, as part of data cleaning, if additional data are required. For example, for radiology data, the HIPS 100 may require what machine was used and all relevant specifications on the machine, the HCP used, etc. Likewise, for bloodwork data, the HIPS 100 may record whether member was fasting or not and collects all specs on the lab, tests, lot numbers, etc. If this information is not available, the HIPS 100 can initiate a process to collect the data. For example, the HIPS 100 may determine, for the health data received from the data source, a health data type, and then, based on the health data type, determine the health data values required for the health data type. This can be done by a look-up table, or a predefined set of rules.

The HIPS 100 then determines whether the health data values in the health data received form the data source do not match the health data values required for the health data type. For a data source for which the health data values of the health data received does not match the health data values required for the health data type, the HIPS 100 determines the health data received from the data source requires cleaning. Such cleaning may involve determining which heath data values that are missing from the health data, and generating a query for the data source requesting, from the data source, the health data values that are determined to be missing from the health data and then sending the query to the data source.

In some situations, the missing health data values may already be stored elsewhere on the HIPS 100 for the member. Thus, before sending the query, the HIPS 100 may determine an index value that references the health data values that are missing from the health data in a health data database (e.g., the user's blood type, for example), and may query in a user s account health data database 110 using the index value to obtain the health data values that are missing from the health data and then augment the health data with the health data values obtained from the health data database.

Turning to FIG. 3, the process 300 may initiate cleaning in a variety of ways. For example, the user may upload personal data 302 to the user account 110, and cleaning check can be instantiated by a cleaning check process 308. Alternatively, the user may identify certain data 306 may need cleaning, or a third party may provide data 304.

The cleaning check process 308 may identify data for cleaning by one or more data integrity checks. For example, missing data values for items can be checked; data ranges, e.g., pulse rates between 50-100 may be accepted, pulse rates outside of this range may cause the process 308 to generate a verification check; etc. If cleaning is required, the user may elect auto cleaning or manual cleaning at 310. For auto cleaning, an auto cleaning process 312, such as described above, is instantiated. If the cleaning decision has high confidence at 314, e.g., a missing data field value that does not change (e.g., blood type, allergies, etc.) can be filled in automatically, etc.) then the data is cleaned at 316, and the history is recorded at 318. Data with low frequency changes or invariable data may be more amenable to automatic cleaning than data that is variable (e.g., weight, blood pressure, etc.). Conversely, if the user selects manual cleaning, a manual process 320 is implemented and the data is then presented to the user for confirmation or rejection of cleaning at 322. The history is recorded at 318. Finally, the user has access to the history to reverse or correct any changes at 324.

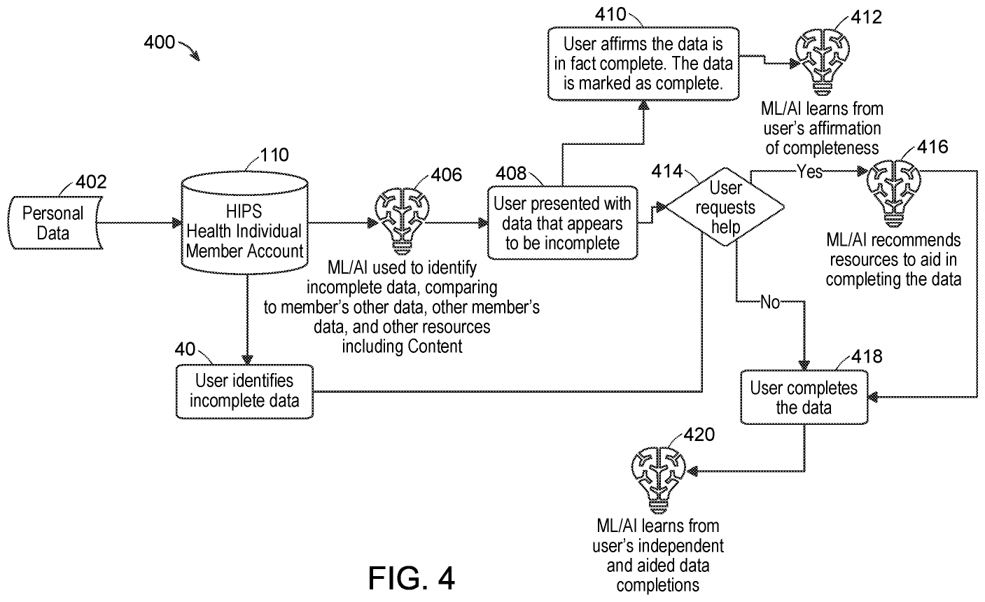

FIG. 4 is a system flow diagram 400 of a data collection process. The process 400 is directed to ensuring data are complete. The HIPS 100 implements, as part of data intake, data validation to ensure data are complete, as described above.

Data 402 are uploaded to be included in the user account 110. A collection check may then be instantiated automatically by a process 406 that automatically identifies missing data, or by the user identifying missing data 404. The process 406 may be similar to the process 308 with respect to missing data field values, etc.

If the process 406 identifies missing data, the user is presented with the data that appears to be incomplete at 408, and if the user affirms the data is, in fact, complete at 410, then in implementations that use machine learning, the process 406 is updated at 412.

Conversely, if the user confirms the data is incomplete, the user may, at 414, either request assistance by a recommendation process 416 to aid in completing the data, or may manually complete the data at 418 if the user does not request assistance. The recommendation process 416 may, for identified missing data, provide prompts to fill in the data, or provide suggestions to find the data, e.g., a suggestion may be “Your blood test results are usually with your hematologist; you can call that provider's nurse line at 404-555-1212.” The contact data can be obtained from the member's data 110, Then in implementations that use learning, the process 406 is updated at 420.

### Data Control

Whether from an EHR, an app, or a device, data is often not accurate. The HIPS 100 provides members with the options to edit data (including removals), annotate data, and submit the changes back to the data source. The IPS 100 can track the changes while surfacing only the updated data. Thus, the member can still access the original data. For data integrity, in some implementations any edited data is flagged to indicate it has been edited. The member can choose to not disclose the original data to other parties, or include an explanation as to why the data was changed. Data recipients (such as HCPs and Beneficiaries) can elect to disregard any data that is marked edited, without the original data. Members may elect to not reveal whether or not changes have been made, in which case HCPs are aware of only that election, and not which, if any, data has been edited, and what the edits were.

For example, a lab shows fasting on one test but not fasting on another; the tests were run at the same time. The user can manually correct the data to show that both tests were subject to the fasting requirements. Other data, such as medications/medicants and dosing schedules, can be manually corrected.

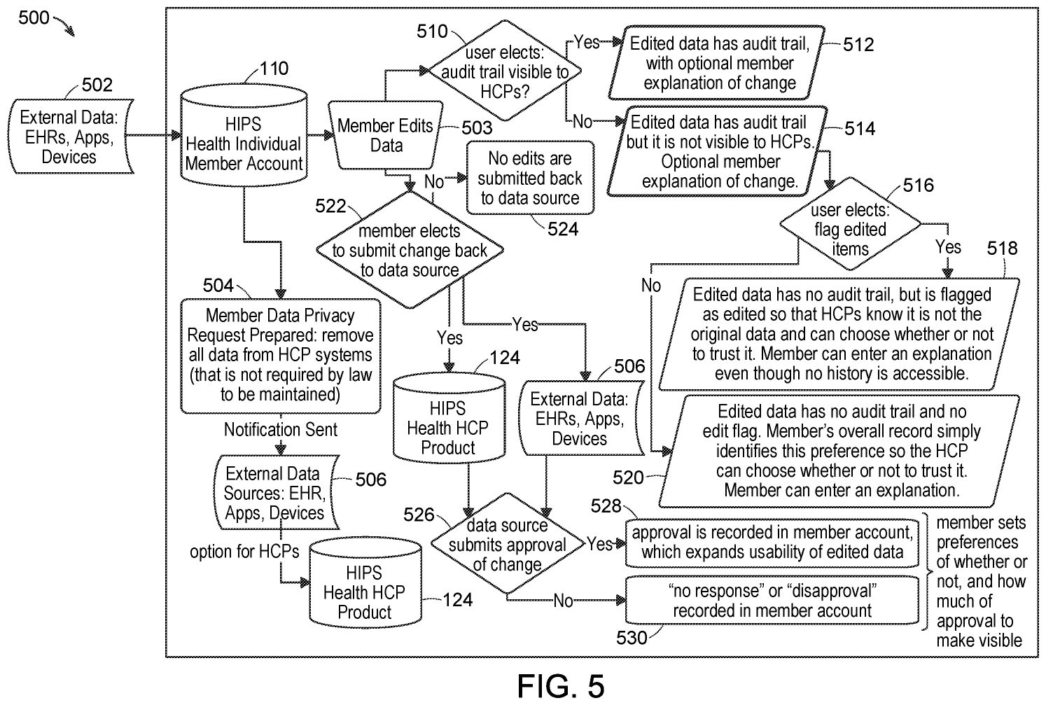

FIG. 5 is a system diagram 500 of a data control process. Data 502 from an external source is provided to the HIPS 100 for inclusion into the member account 110. Data control can then be exercised in a variety of ways. For example, at process step 504, the member prepares a request to remove data from an HCP third party system. The request is sent from the HIPS 100 to the third party external source at 506, and processed by the third party within their HCP third party system or using the HIPS HCP product 124 (described in more detail in the following sections).

The member may also edit the data at 503. After editing, the user may elect whether the edits/audit trail is available to the HCP. If so, the HIPS 100 ensures the edited data has an audit trail with optional notes at 512. If not, the audit trail is not available to the HCPs at 514.

If the user elects to flag items at 516 after not electing to make the audit trail visible, then at 518 the HIPS 100 flags the edited data to indicate to the HCP that the data is not original. The member may provide an explanation as to the edit.

Conversely, if the member does not flag the items at 516, then the edited data has no flag and no audit trail at 520.

The member at 522 may elect to submit the changes back to the data source at 522. If the member elects not to do so, then at 524 the edits are not submitted back. Conversely, at 526, the data source receives the edits for changing at their own data storage. The data source may then approve the change, which, if done, results in approval recordation at the HIPS 100 at 528. Conversely, if the data source does not respond or disapproves, then at 530 the non-response/disapproval is stored in the HIPS 100 member account 110.

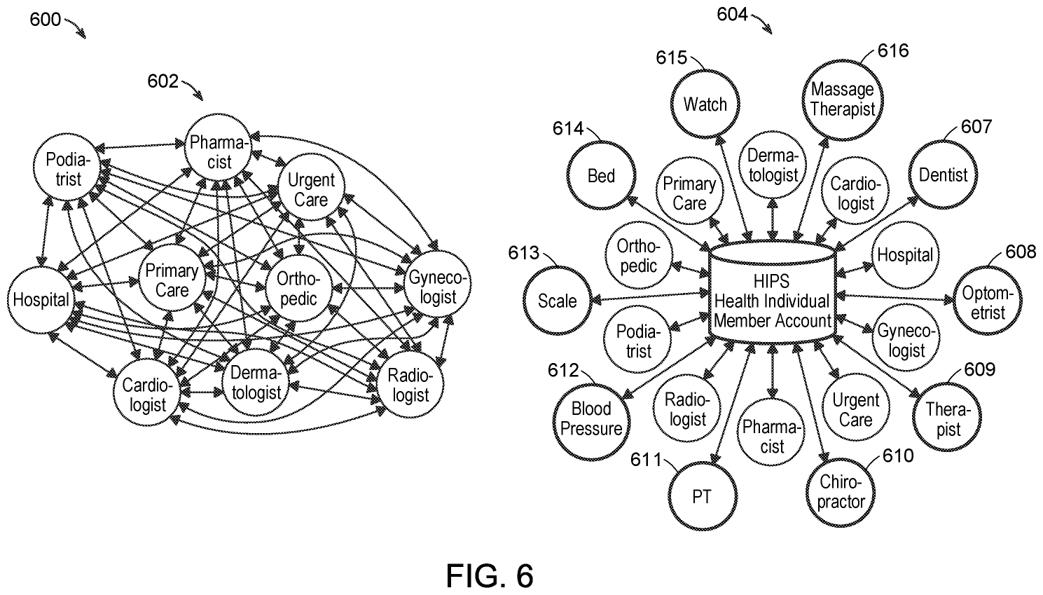

FIG. 6 is a diagram 600 of system interoperability. The diagram 602 illustrates current data management without use of the HIPS 100, and the diagram 604 illustrates the centralized management of user data and additional data by use of the HIPS 100.

Diagram 604 illustrates the improvement over existing systems. The HIPS 100 makes use of additional third-party data sources, e.g., 607-616, and provides interoperability that enables member's data to be easily imported to their HIPS account 110. The HIPS 100 also enables member's own elections to choose which data sources from which to import their personal data, along with which of its third-party data sources it wants to allow to receive data from their HIPS account 100.

For example, consider a member has 10 HCP portals. Each HCP has collected data about the member and has successfully recorded that data in the EHR. Each of the HCPs offers full interoperability (or, if not, the HIPS 100 enables data cleaning to store in standardized formats, as described above), allowing the member to activate HIPS to import all the data from each HCP, into the member's HIPS account. When a member has elected full interoperability with all HCPs, the HCPs can then each access the member's full HCP records by use of the HIPS HCP product 124, described in later sections below,

### Credentials and Logins

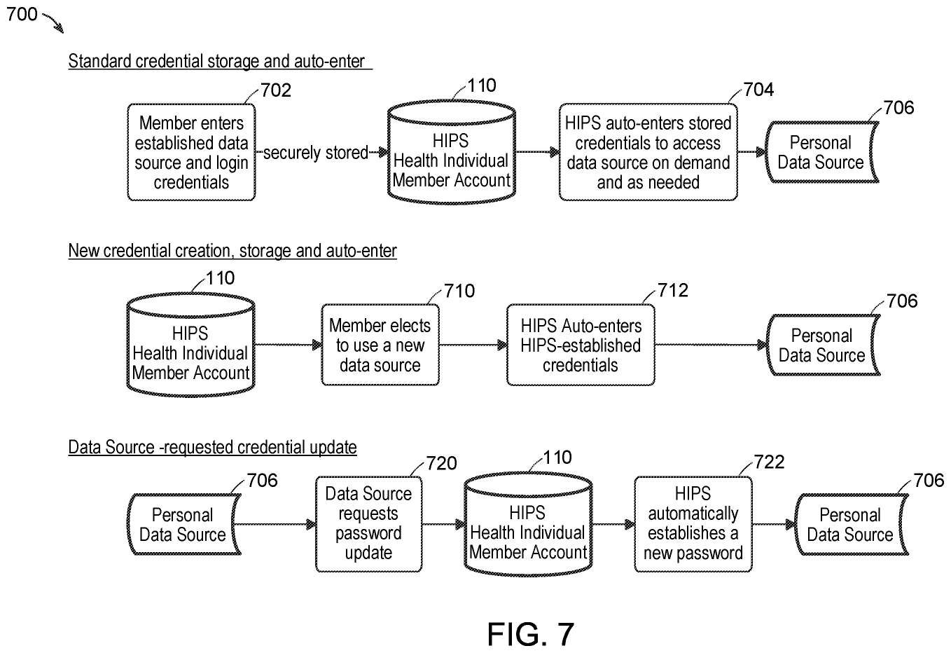

FIG. 7 is a system flow diagram 700 of credential processing. The HIPS 100 implements security protocols to manage access to a myriad of member portals, devices, apps, etc., which are referred to as personal data sources 706 in FIG. 7. The HIPS 100 creates accounts and sets up credentials for new personal data stores 706 and stores and maintains access to the data sources 706 for the member account 110. This eliminates the need to create and maintain logins for tens or more of different personal data sources 706. Credential management depends on particular uses cases.

For example, when the member accesses HIPS account 110 at 702, the system automatically accesses login information the member has provided for each personal data source and auto-enters that information at 704 for each required data sources 706, granting seamless access from HIPS to the personal data sources 706.

When the member gets a new HCP, new app, or new device at 710, the HIPS 100 does a new credential creation process. The HIPS 100 creates a HIPS credential for the new data source by automatically creating and setting up the login credentials on behalf of the member, without the need for the member to store that information, and optionally, even know what the credentials are.

When the data source 706 initiates a request for a credential update, as may be required periodically, the HIPS 100 automatically receives the request, creates the new credential at 722, and submits the update to the data source 706.

An example security credential process is thus as follows. For each of the users, the HIPS 100 establishes, for the user, a unique account. The HIPS 100 then receives for the user, a set of data sources for the user, each data source being different from each other data source, and each data source requiring login credentials for the user. For each of the data sources, the HIPS 100 establishes, without user input, login credentials for the user, and stores, in association with the unique account for the user, the login credentials for the user.

Thereafter, for at least one of the data sources of the set of data sources for the user, the HIPS 100 establishes, using the login credential for the data source established by the system, a connection to the data source, and then receives, from the data source, health data specific to the user and stores the health data in the unique account of the user.

### User Experience Customization

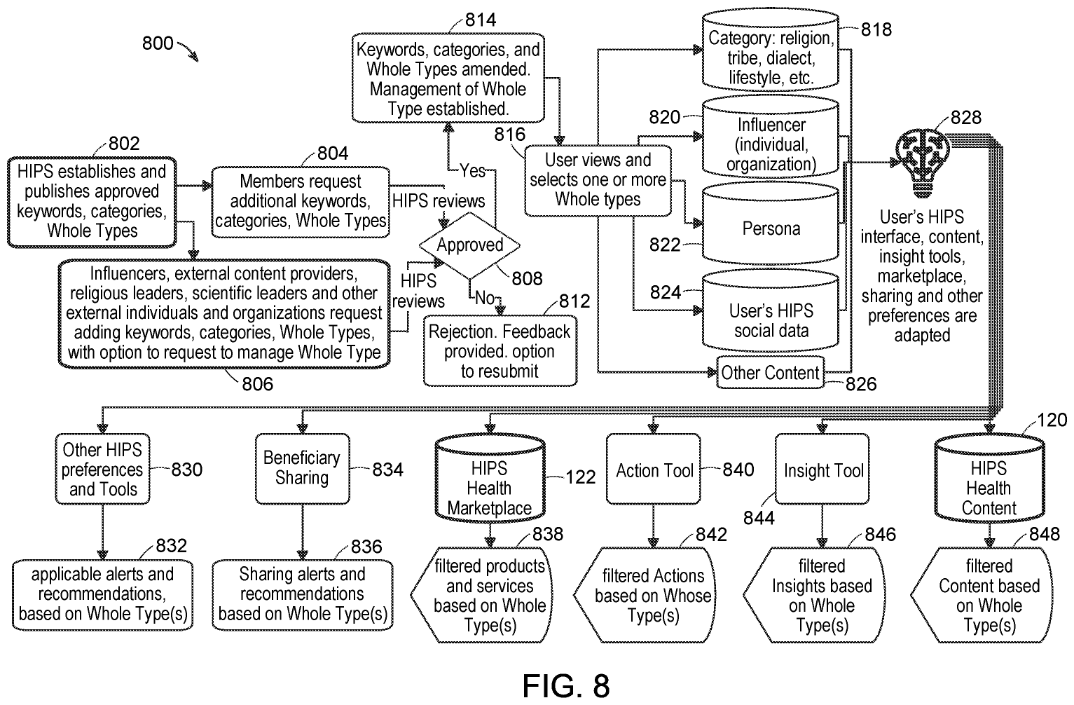

FIG. 8 is a system flow diagram 800 of user experience customization. The customization 800 allows users to experience the benefits and recommendations of the FHPS 100 based on the user's user experience customizations. The experience customization 800 allows members to select a variety of types that they relate to best. An individual may relate to a HIPS persona, an influencer or celebrity, a religion, a culture or tribe, or a particular level or type of scientific rigor or belief. The user can express these preferences and the HIPS 100 can generate a set of weights by which user recommendations and other information are selected for presentation to the user. These designation preferences chosen by the member customize their experience for the whole of the HIPS 100 system tools and services, and are generally referred to as “types” or “whole types.”

For example, for each user, the HIPS 100 determines from the health data stored for the user account 110 of the user, health preferences. These preferences may be selected by the user. The HIPS 100 then determines, from the health preferences, a set of type weights, wherein each type weight describes an estimated level of user interest in a particular health service type. For example, one user may have a strong preference for homeopathic treatments, while another may prefer only treatments approved by regulatory agencies. Based on these type weights, the HIPS 100 selects one or more health service recommendations for the user and provides the recommendations to the user. Such recommendations can include adjusting share levels of health data, a health product recommendation, a health action recommendation, and a health content stream recommendation.

In operations, HIPS 100 publishes keywords, categories and types at 802. Users may request additional keywords and types at 804, e.g., a particular user may desire to have a particular interest category includes. Likewise, external parties that users may follow may also request particular information be included, as shown in 806. The HIPS 100 system then reviews (either automatically and/or by human curation) and approves or disapproves. If disapproved, then a rejection notice is sent at 812.

If approved, the keywords, categories and types are updated at 814. Thereafter, users may select types for user experience customization. The types may include cultural categories 818, influencers 820, a persona 822, the user's HIPS social data or other social data 824, and other content 826. Based on these preferences, the user's interface, content, insights, marketplace and other information are adapted at process 828. The process 828 can select default displays and options based on preference sets, or, alternatively, can query the user for certain changes and request confirmation by the user. Thus, for a given preference set, the process 828 can select corresponding interfaces, insights, tools and the like. A rule set can be used to make the selections, or a machine learned process can be trained over time to make changes for preferences.

Personas 822 are personas a member may select, e.g., BETSY: Middle-aged white working mom, high blood pressure, high cholesterol, moderate exercise; JOE: Single mixed African American/Caucasian male, mid 20s, athletic, high school diploma, working in service industry, etc.

The influencer 820 can be a celebrity, role model, or a more modern social media influencer. The HIPS 100 categorizes each available candidate according to content, e.g., sports/basketball, cosmetics/hair products; etc.

Based on the specified and derived preferences, the HIPS 100 then customizes the user experience for each of the HIPS 100 services. For example, for general preferences and tool 830, applicable alerts 832 are generated and adjusted. For beneficiary sharing 834, sharing alerts and recommendations 836 are generated. For the HIPS marketplace 122, filtered products and services 838 are provided. For action tools 840, filtered actions 842 are provided. For insight tool 844, filtered insights 846 are provided. For HIPS health content 120, filtered content 848 is provided.

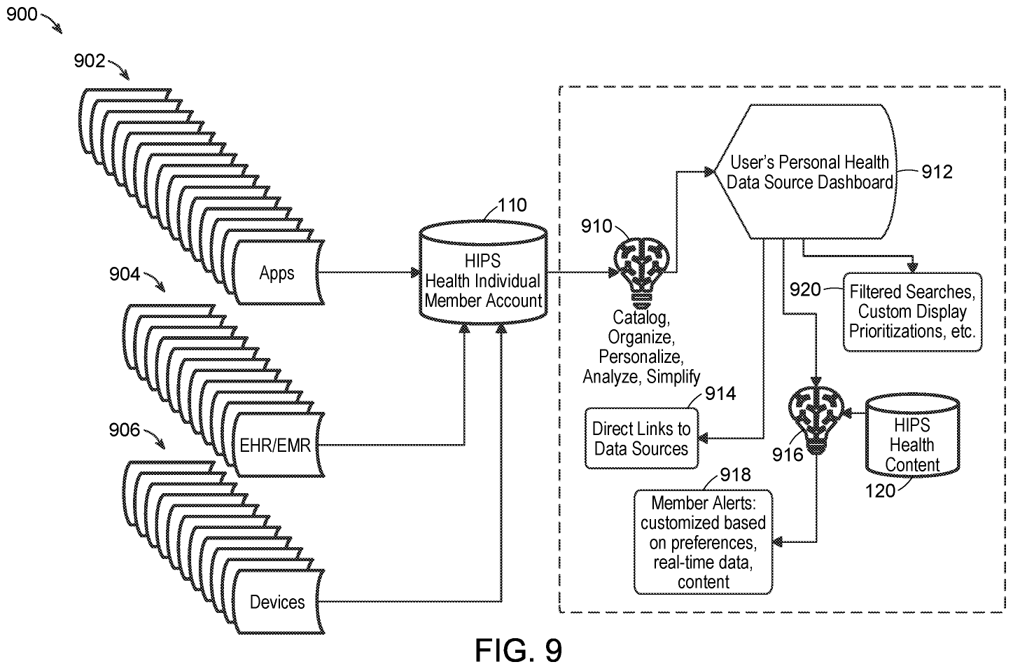

FIG. 9 is a system flow diagram 900 of data organization. The data organization 900 can be implemented by means of a dashboard environment 912. For a user account, all data sources 902, 904, and 906 that contribute to and/or interact with the member's HIPS account 110 are processed and cataloged at process 910 for concise presentation for the member. Any appropriate indexing and categorization process can be used for process 910.

The dashboard 912 includes links 914 to the data sources 902, 904 and 906. These can include portal links, app links, and other links that are available from the data sources.

The dashboard 912 can also show the results of alert process 916 in the form of member alerts 918. These alerts are customized based on preferences, data, etc. For example, a member may have an alert that notifies him when pollen counts exceed a level. The level may be determined based on his medications, history, and health content 120, etc. The member may adjust the alerts accordingly. Alerts may be selected by 916 based on a rule set, or may be learned by a machine learning process.

Filtering and display options 920 may also be displayed to the user, and the user may also adjust these accordingly.

### Data Sharing

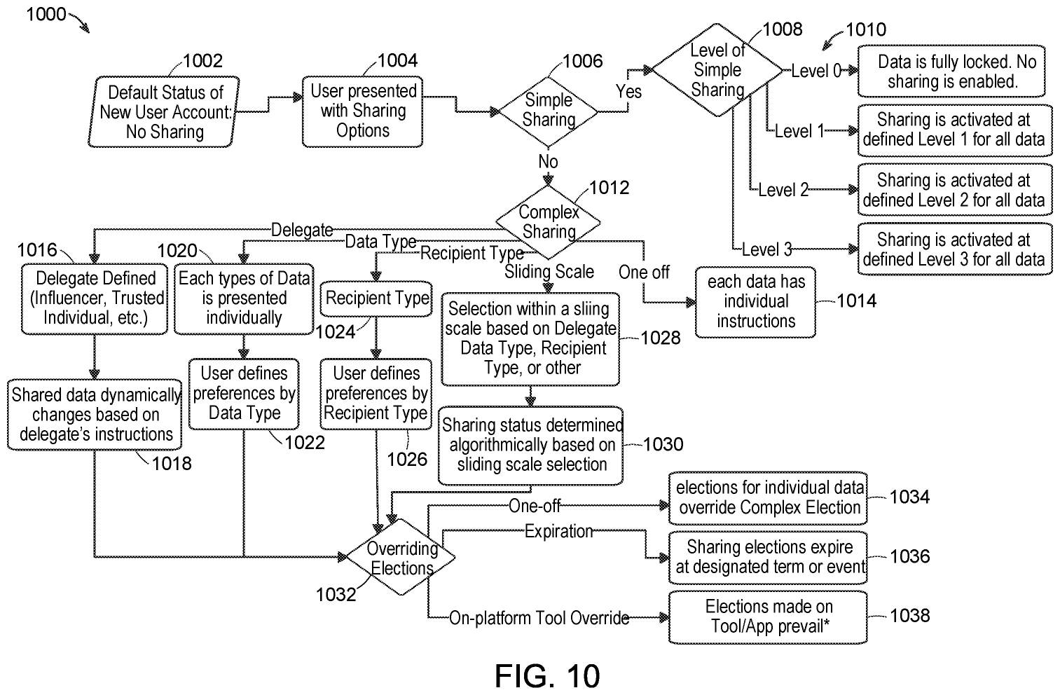

FIG. 10 is a system flow diagram 1000 for global sharing preferences. The HIPS 100 allows users to implement sharing schemes ranging from the very simple to the very complex. The HIPS 100 provides members with the ability to share their aggregated HIPS data with beneficiaries should the user choose to do so. Sharing is done according to member specified terms with ongoing transparency of the use of the data. Beneficiaries are any individual or organization that will potentially benefit from receiving the shared data. It includes individuals such as caretakers, loved ones, and HCPs, and organizations such as payers, researchers, product and service providers and government.

A simple sharing option allows selecting from popular sharing options across various levels of sharing. Conversely, a complex sharing option can involve assigning a delegate such as an Influencer or trusted person, defining preferences based on the type of data or type of recipient, selecting options from a sliding scale, and/or providing individual instructions for individual data. Overriding elections can be designated at any time, whether for a specific circumstance, until a designated time or event, an election made on an app or HIPS 100 tool.

One example sharing option is a standard share option. HIPS 100 provides this default for simple recommendations on what to share with whom, based on who the beneficiary is, what is relevant in the current situation, and what they are requesting.

Another example sharing option is a share all option. The member gives full authorization to share everything with everyone without beneficiary proposal review. Beyond the restrictions and deidentification (anonymization) protocols implemented by HIPS 100, all member data are eligible to be shared with qualified beneficiaries.

A member can override “All” with limitations to specific types of companies, for example: (a) only to review new devices, or (b) only to help with oncology research, etc. A member can also allow requests for additional data collection. Thus, a beneficiary that is interested in a member's data can query for data collection.

Depending on the share options selected by a user, an HCP may be granted access as view only, full sharing with download/copying capabilities, or anything in between. This allows members to retain the security of HIPS 100 without distributing the data but also allows the flexibility to push the data off HIPS 100 to provide the HCP with potentially greater utilization of the data.

Copy access may be limited as well. A full copy option allows the HCP to copy the member's data directly to their EHR. Conversely, a no copy option allows the HCP to simply view the data without copying it to their EHR.

More generally, the HIPS 100 receives, from the user of the account, share data specifying a plurality of share levels for the health data, wherein different portions of the health data are each associated with a different share level. For each portion of the health data associated with a particular share level, the HIPS 100 enables sharing of the health data according to the share level.

Sometimes users may opt into clinical trials or other obligations to share data. The HIPS 100 has a process to ensure that the share levels are consistent with the obligations. For a user account, when the HIPS 100 receives data describing a user obligation to share particular health data of the user with a third party, the HIPS 100 determines whether the share level of the particular health data precludes sharing of the particular health data with the third party. If the share level of the particular health data precludes sharing of the particular health data with the third party, then the HIPS 100 determines a user preference for a share conflict, and performs a share resolution process according to the user preference.

The user preference may specify an automatic override of the share level. For this preference, the HIPS 100 allows, without user confirmation, sharing of the particular health data with only the third party. Otherwise, user confirmation is required.

As shown in FIG. 10, the default status 1002 of an account 110 is set to no sharing. The user, by means of a menu or command, is presented sharing options at 1004. The user may select simple sharing 1006 or complex sharing 1012. If simple sharing 1006 is selected, then, in some implementations, a predefined set of sharing options indicated by a level of sharing is selected 1008. Four example levels 1010 are shown, from 0 (No sharing) to 3. Generally, the higher the sharing level, the more data that can be shared and the fewer alerts and safeguards that are enacted.

If the user selects complex sharing 1012, then the user may select a one off option 1014. In the one off option 1014, the user may specify sharing instructions for each data set, e.g., blood work may have a first sharing option, oncology results may have a second sharing option different from the first, etc.

Other complex sharing options can also be selected. For example, a user may set a delegate 1016 for sharing data, and the delegate may also control sharing of the data at 1018. The user may also share by data types 1020, and set preferences for each data type. The user may also select sharing based on recipient type (e.g., HCP type, family type) and set sharing preferences based on the recipient type.

In other implementations, a sliding scale UI tool 1028 may be used to set sharing types, where each scale positons has a predefined set of sharing preferences associated with it or the preferences are determined algorithmically 1030 based on the scale position.

Once set, preferences can be overridden at 1032. The override may be a one off 1034, an expiration 1036, or an on-platform tool override 1038. For the one-off 1034, the elections for individual data override the complex elections. For the expiration, the sharing preferences expire at the occurrence of an event (e.g., a time). For the tool override 1038, a new set of complex options are received and replace the prior set.

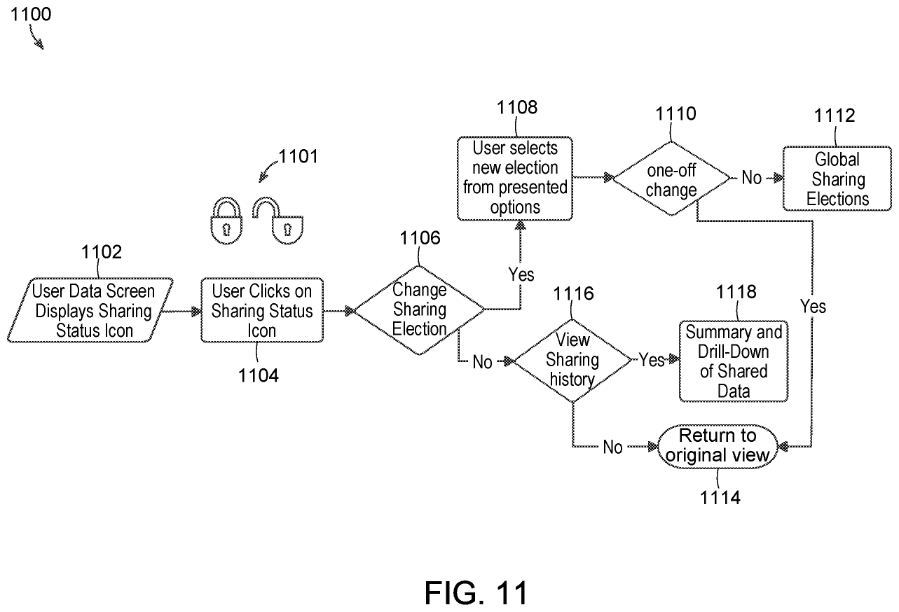

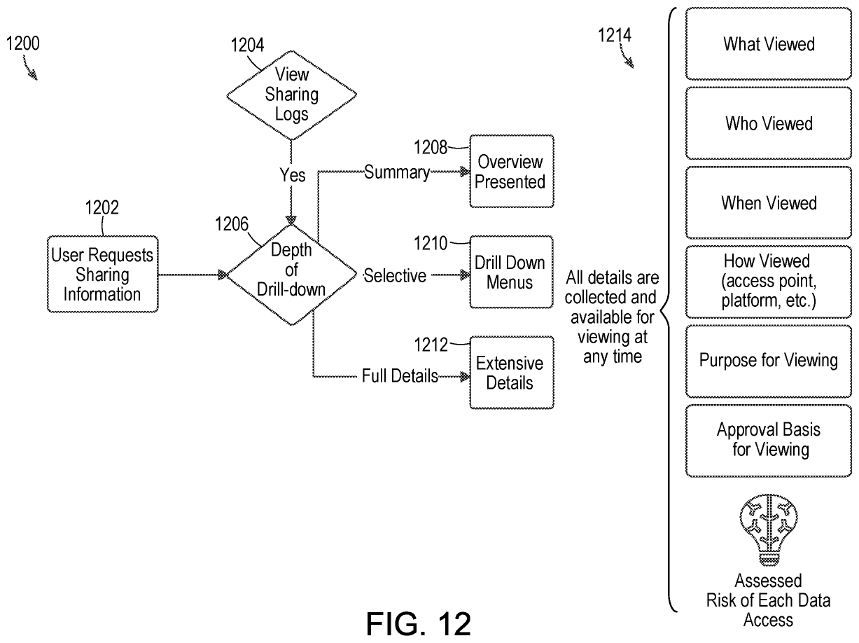

FIGS. 11 and 12 is a system flow diagram 1100 and 1200 of adjusting sharing preferences. In some implementations, sharing functionality is implemented by use of lock/unlock icon 1101. In a screen that displays the icon 1101, the user may select the icon 1101 to toggle from a locked to unlocked state. When unlocked, sharing preferences may be altered. If a user changes an election/preference at 1106, then at 1108 a user selects a new election from presented options.

The HIPS 100 determines if the change is a one off at 1110. If not, then at 1112 global sharing preferences are set. If the change is a one off, then the process is complete and the interface returns to the original view at 1114.

If no change is selected at 1106, then the user has the option to view sharing history 1116. If the user chooses yes, then at 1118 a summary and drill down of shared data is shown. Otherwise, the interface returns to the original view at 1114.

The HIPS 100 stores complete logs of all member data shared historically. As shown in FIG. 12, when the user requests access to the logs at 1202, or to view sharing logs at 1204, the user is presented with the option for drill down depths 1206. The drill downs may be a simple overview 1208, selected drill downs 1210, and extensive details 1212. The logs indicate many sharing features 1214, such as who accessed the shared data, when they accessed, how the data was viewed, the purpose for viewing, the approval basis for viewing, etc. The logs may also include links to legal agreements that grant sharing of device/app data.

The HIPS 100 includes an alert process to notify the member of repercussions of locking data that was previously unlocked, including in particular initial “Share All” elections. These may include HCP sharing that is needed for the agreed upon HCP alerts and/or monitoring of the member's vitals or data sharing agreements with beneficiaries.

### Data Summarization in Story Form

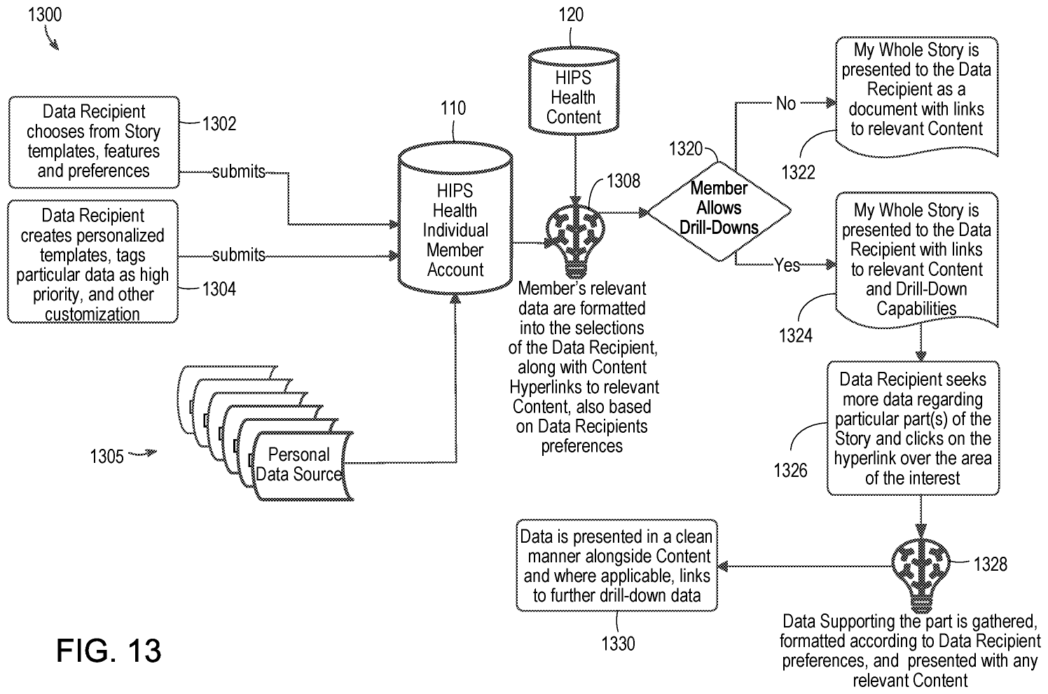

FIG. 13 is a system flow diagram 1300 of a data compilation process in story form. The HIPS 100 can produce data on demand with a compilation of relevant sets of data, presented in a variety of ways, with customization available by the member and by the data recipient (HCP, etc.). In some implementations, the data compilation can be a written narrative form, with relevant details that have been compiled in the HIPS 100 from the member and from all data sources.

As shown in FIG. 13, a data recipient can choose from a set of predefined story templates 1302 or can create a template 1304, and the templates are submitted to the HIPS 100. When a template is selected, the member's relevant health data (e.g., data in the member 110, which can be data from data sources 1305, including data from FHIR, data monitoring devices, and user inputs) is selected by the HIPS 100 process 1308 along with content links to relevant health content 120 (the latter may be, for example, clinical studies and journal articles that may be relevant to the user's condition.) The process 1308 may, for example, compare field data in the templates to the index values in the member data 110 to generate the data for presentation.

Depending on preferences of the member, the story may or may not allow drill downs 1320, If drill downs are not permitted, then at 1322 the story is generated as a document with links to relevant, public health content. Conversely, if the drill downs are permitted, then at 1324 the story is presented with links to the public content and drill-down links to the user's health information. Should the data recipient request more data at 1326, then the process 1328 (subject to sharing requirements) collects the additional data and the additional data is presented at 1330.

Example templates include an incident template and an emergency medical access template. The incident template allows for generation of an automatic compilation of all background data relevant to a particular incident (e.g., migraines), up to, during, and following an incident. The emergency medical access template allows for generation of a report that highlights general information such as allergies and medical conditions, and may automatically authorize temporary access to authorized medical emergency HCPs in the event of an emergency where time is critical and member authorization is impossible or disruptive to their care. It provides instant access to the member's full HIPS records, provided the member has agreed to allow the report beforehand.

### Input Tools

The HIPS 100 utilizes a number of different input tools for data collection not only from HCPs but also from third parties. These include a baseline tool, and comprehensive data analysis tool, a prescription and supplements (“medicants”) tool, a transcription tool, and other tools, which are described below.

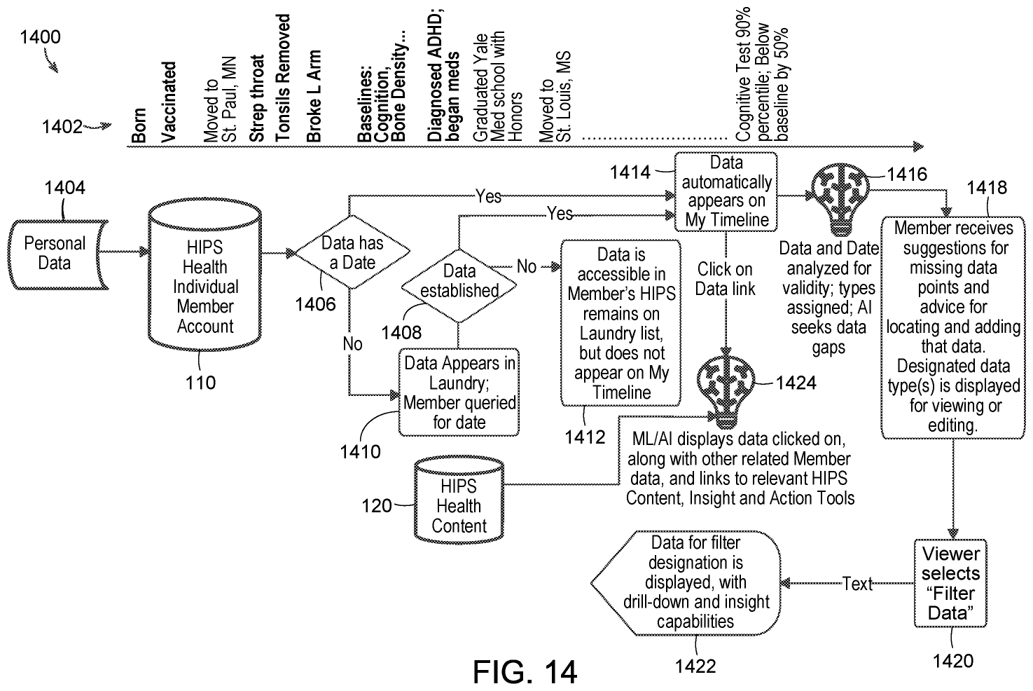

FIG. 14 is a system flow diagram 1400 of timeline processing for a timeline tool. The HIPS 100 can summarize, sort and present data according to time tags. The data includes both health related data and non-health related data, such as location, events, etc. The HIPS 100 includes drill-down capabilities and filtering. Members may provide data in addition to data provided from EHRs, devices, apps, and other systems.

The non-health centric data can include any data describing activities or events that impact our health but is rarely part of a medical history, such as marriage, children, pets, companions, locations lived and visited (all travel), schools attended, education (including performance and accomplishments, helpful in tying together many data points), lifestyle data, relationship and potential direct records from blood relatives (similar to the typical family history, but with far more information), weather and other events that may affect health.

In some implementations, the HIPS 100 receives, from one of the data sources, a health history request for a user. The health history request specifies a plurality of health data values of the user to be provided. In response to the health history request, the HIPS 100 provides to one of the data sources, the health data values of the user. This may be useful, for example, when a user goes to a new provider. If the provider has a HCP account on the HIPS 100, the provider can receive the new patient health history data to auto-populate the provider's health history form and also have the necessary data imported for evaluating the new patient. In some implementations, the HIPS 100 system sends a confirmation request to the user to provide the information, e.g., a text message that reads “Doctor X has provided a health history request. Do we have your consent to provide this information?” If the user selects Yes, and the information for the form is auto-populated.

The user's health history information is shown in timeline form 1402 in FIG. 14. As data 1404 are provided to the user account 110, the HIPS 100 determines if the data has an associated date at 1406. If no date is provided, then at 1410 the HIPS 100 queries for the date as part of the laundry process. If the date is still not established, then the data is marked as incomplete but available to other tools besides the health history timeline at 1412.

If the data does have a date or one is provided, then at 1414 the data can appear on the timeline. It may optionally be analyzed for accuracy at 1416. Any appropriate data accuracy or integrity checking process can be used. At 1418, the member may receive suggestions for missing data and locating the missing data. At 1420, the viewer can filter the data, and then at 1422 the data as filtered is displayed. Drill down and insights may also be available.

Should the user select data that is displayed, the process 1424 causes the data to be displayed to the user, e.g., by a HTTP request, or any other appropriate data request mechanism, and may generate additional data for display to the user, using one or more data relevance/search processes.

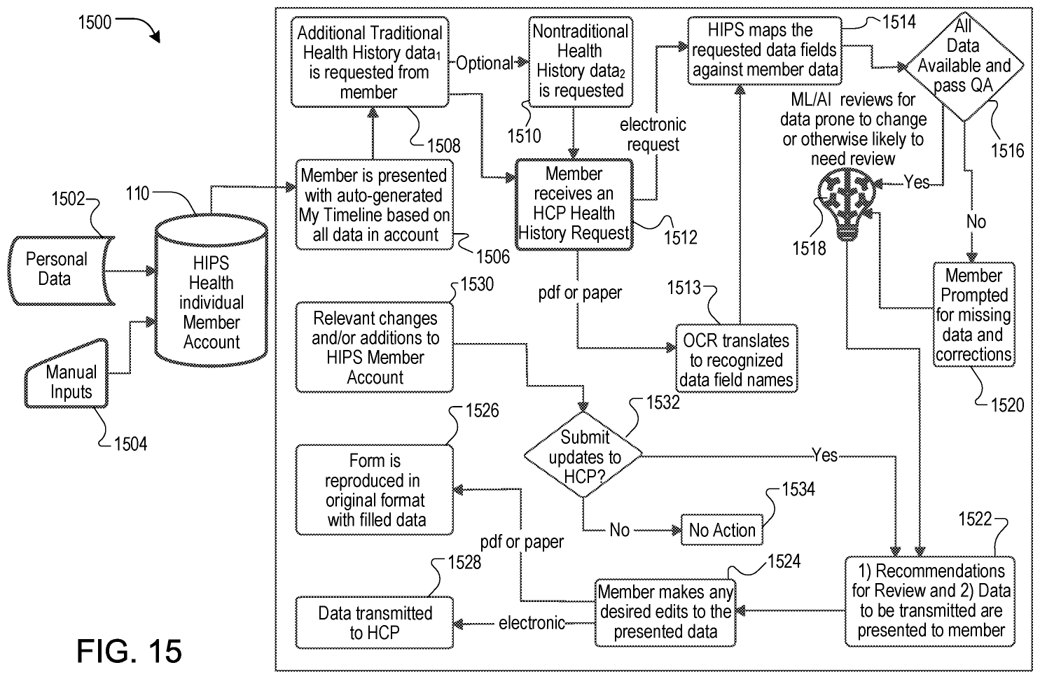

FIG. 15 is a system flow diagram 1500 of health history processing. The health history toot of the HPS 100 automatically generates an up-to-date health history, on demand, to share with new HCPs and with existing HCPs who routinely request updates. The IPS 100 provides complete, accurate, and non-repetitive data for the member, to assist in collecting all data being sought by the HCP.

As illustrated in FIG. 15, the member account data 110 includes data from personal data sources 1502 and manually input data 1504. At 1506, the member is presented with the auto-generated timeline data based on data in the member account 110. Additional data, such as other data from the member's health history 1508 and non-traditional data 1510 may also be requested.

At 1512, the member receives the health history request. The request can be electronic or in paper. If electronic, e.g., a new patient is providing data though an HCP portal, the HIPS 100 maps the requested data fields against the member data at 1514, and then determines if the data is available and passes quality assurance in 1516.

If not electronic, then at 1513 the paper undergoes OCR to convert to electronic, and the process continues at 1514.

If the data is available and passes quality assurance, then at 1518 the HIPS 100 reviews the data to determine which data are prone to change or will otherwise need review. Data that are prone to change are data that, based on history, vary at least by a frequency value, and the process 1518 can check for such data. Conversely, if the data does not pass quality assurance, then the member is prompted at 1520 for missing data.

At 1522 recommendation for review and data to be transmitted are presented, and the member at 1524 makes any necessary edits. For paper forms (or electronic documents), the form is reproduced with filled in data at 1526. Otherwise, the data are transmitted to the HCP at 1528.

If changes of data occur at the member account 110, as shown in 1530, then at 1532 the HIPS 100 determines whether to transmit the changes to the HCP. If so, the process resumes at step 1522. Otherwise, no action is taken at 1534.

### Baseline Processing

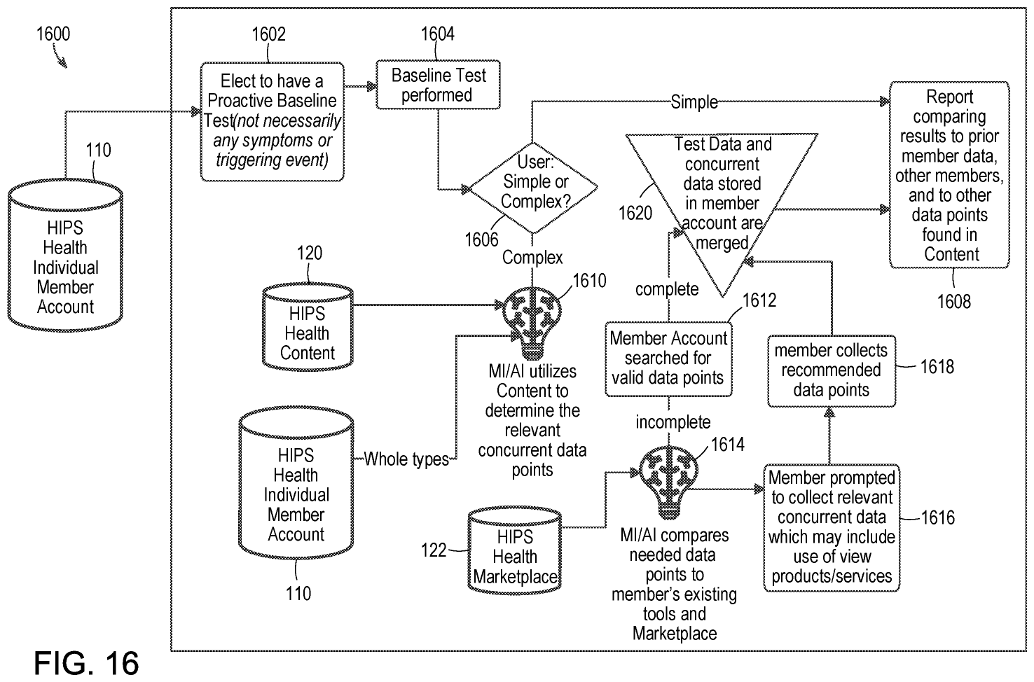

FIG. 16 is a system flow diagram 1600 of processing data against baselines. The HIPS 100 includes a baseline evaluation tool that provides members the ability to track their baseline health measurements over time. The tool allows for an initial baseline and ongoing baselines over time. Examples of common progressive baselines screenings are standard blood tests, bone density tests; genetic testing and tracking epigenetic changes, and cognitive testing.

In operation, the HIPS 100 determines a baseline value for health metric value of a user. The baseline value can be determined in a variety of ways, such as a last test result, an average over time, or some other technique. The HIPS 100 then determines if the metric value at a later time, based on the health data of the user, has deviated from a baseline value. If so, the HIPS 100 system determines one or more health inquiry questions to present to the user. The questions are then presented, and the responses stored. Based on the responses, a potential cause that caused the health metric value to deviate from the baseline value is determined.

For example, a person may have gained twenty pounds from a last physical. The HIPS 100 determines the weight has deviated from the baseline value, and inquires of the user what may have caused the change. Questions such as “You have gained 20 pounds since your last physical. It appears that your exercise volume is also down. Have you not been exercising?” Depending on the answer, other questions can also be asked. The questions can be selected from a variety of diagnostic tools.

In FIG. 16, the baseline tool, in response to a proactive baseline test election 1602, performs a baseline test 1604. The test can be performed by the HIPS 100, such as a cognitive test, or can be performed by an HCP and the results provided to the HIPS 100. The test can be simple or complex as specified at 1606. A simple test generates a report 1608 comparing to prior member data, other members, and other data found in the health content.

A complex test may be more involved, and depend on the type of test being performed (e.g., cognitive testing, etc.). The HIPS 100 implements a process 1610 to determine relevant data points for the test, and then searches the member account 110 and health content 120 for valid data points to perform the testing at 1612. Process 1610 can select the relevant data based on a rule set, or based on a machine learned process. If the data are present and valid, step 1620 conducts the test and the report 1608 is then generated.

If the data is incomplete, the process 1614 may determine that additional services or devices are required. For example, the process 1614 can be a process that is programed to determine, based on a missing data set, tools or devices that can allow for monitoring or provisioning of such data. The process 1614 can be rule based or machine learned. For example, the person may need to provide daily blood pressure readings, but does not have a blood pressure meter. The HIPS 100 may access the health marketplace 122 to recommend a blood pressure meter for the user at 1616 for data collection. After the user secures the service or goods, the data is then collected at 1618, and then 1620 is then performed.

### Comprehensive Data Analysis

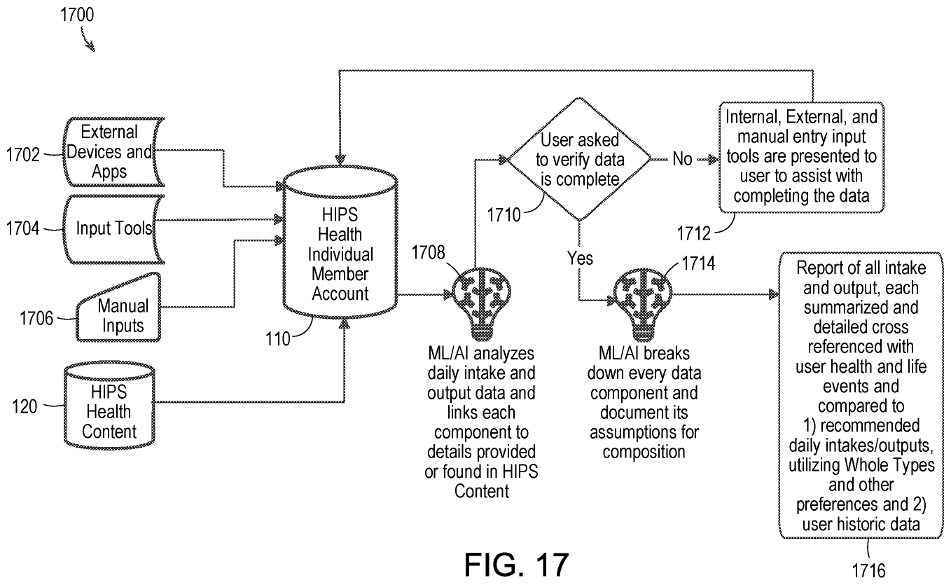

FIG. 17 is a system flow diagram 1700 of processing multiple inputs for a comprehensive analysis. The HIPS 100 comprehensive analysis tool processes health data from a variety of inputs, including manual inputs, prescriptions and supplements, etc., and provides recommendations for achieving one or more goals, e.g., vitamin absorption, improving any concerning blood results through changed lifestyle, adapting food and drink and/or improving adherence to prescriptions and supplements.

As shown in FIG. 17, the member account 110 includes data from external devices and apps 1702, as described above, data from input tools 1704, manual inputs 1706, and health content 120. The HIPS 100 implements a process 1708 to analyze daily intake and habits and determines relevant data for achieving a goal. The process 1708 can be rule based or machine learned to determine, for a goal, the data to be monitored.

At 1710, the user is asked to verify that the data is complete. If the data is not complete, e.g., the user has forgotten to input medical prescription intake, or has forgotten to report a test result, etc., the user is prompted to provide the data and provided one or more tools to do so at 1712, If the data is complete, then at 1714 the HIPS 100 implements a process to generate a report at 1716 that reports intake and outputs, cross referenced with user health and life events, and provides recommendations to the user. The process 1714 can be a rule based process or a machine learned process to generate the desired output. The recommendations take into account the user's preferences and are tailored to assist the user in achieving one or more goals.

### Medicant Support and Management

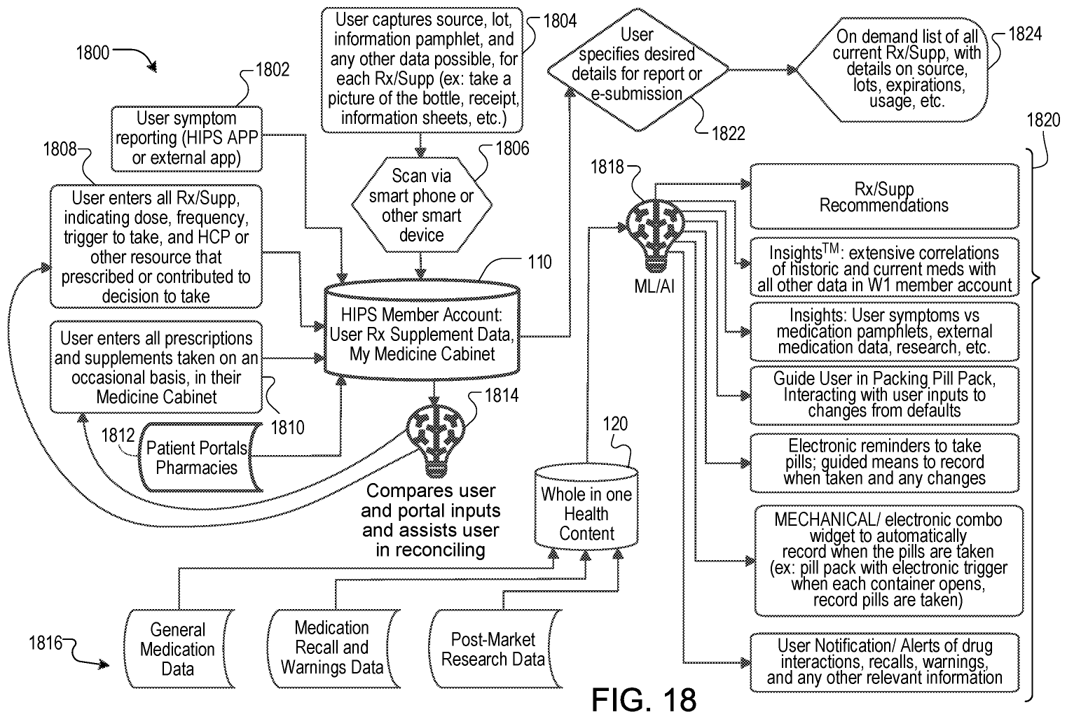

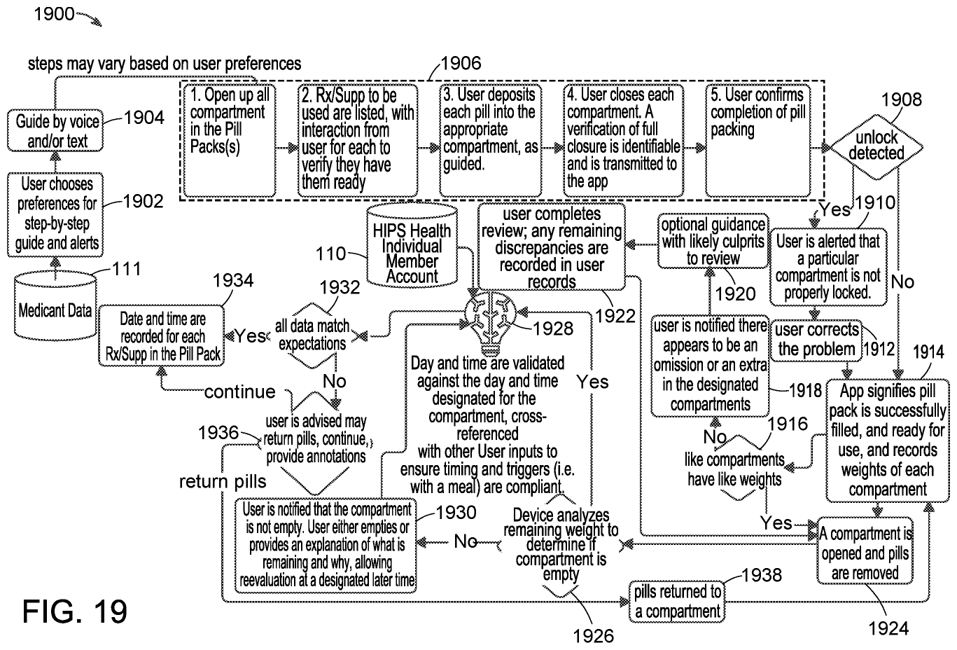

FIG. 18 is a system flow diagram 1800 of detailed tracking prescriptions and supplements. This information may be used in providing dosage intake schedule recommendations to the user, and for administering an electronic pillbox, as described with reference to FIG. 19.

Detailed tracking of prescriptions, over the counter medicines, and supplements (all generally referred to as “medicants”) taken by an individual is typically only performed in an inpatient setting. Adherence is increased by guiding the filling of pill packages, tracking what is put in the compartments, and providing reminders for taking the pills. Tracking the time taken is important data to maintain and powers alerts to family and/or caretakers if the member is late in taking an important medication. Maintaining records of the pill lots also is advantageous in case there is variance in pills from one lot to the next, for immediate notification of recalls, and for supporting population wide feedback on efficaciousness of different suppliers, lots, etc. The HIPS 100 implements processes to track a user's medicants, determine an optimum dosing schedule, and determine, by either manual prompting or by use of an electronic pill package, whether the user is adhering to the dosing schedule.

Inputs to the HIPS 100 medicant management process includes member symptoms 1802, source and lot information 1804 of medicants, and other health data and events that are already being collected, whether within a HIPS 100 tool or within an external data source.

Users may enter the current dose, frequency, and trigger (such as with breakfast, symptom-triggered, etc.) for each medicant, as well as the prescribing HCP at 1808. They may also enter medicants taken on an occasional, as-needed basis at 1810. The data on the bottle or package may be scanned (as indicated by 1806), or otherwise submitted, by the user. Each time a new container is opened for use, that information is updated, along with the start date, by the user. Another source of data is a pharmacy portal 1812 that can provide the information to the HIPS 100.

At 1814, the HIPS 100 implements a process to ensure that the user and portal inputs are consistent, and, if necessary, requests reconciliation by the user. The process 1814 can be a rule based process or a machine learned process to generate the desired output. Any appropriate data consistency evaluation process can be used.

Once the data are input, the user can quickly specify desired details to report at 1822, and the HIPS 100 generates at 1824 a list of medicants and information with full details available including source, lots, expirations, usage, and any other details specified by the member.

The HIPS 100 also implements a recommendation process 1818 that takes as input data from the user member account 110 and the health content 120 and provides multiple recommendations 1820. The process 1820 can be a rule based process or a machine learned process to generate the desired output for the set of inputs.

One type of recommendation is personalized dosing and timing. The recommendation is based on weight, blood type, metabolism, prior reactions, other meds being taken/interactions timing with food and meds for best absorption, genetic tests, etc. The process 1818 cross references this data with health content 120 and provides personalized dosing and timing recommendations. Additionally, a pharmacist and doctor can also utilize all this information to make the best recommendations for OTCs, as well as more personalized dosing for prescriptions medications.

Symptoms and side effects can also be summarized for the user for easy reference. Additionally, interactions and recalls can be provided to the user in alert form.

Electronic data can be provided to an electronic pill package or other device to remind the user to take medications at particular times, and request verification that the medicants have been taken. The recommendations 1820 can also provide aid in replenishment, recalls, new research warnings/notices, etc.

FIG. 19 is a system flow diagram 1900 for pill package medical device processing. The flow diagram 1900 is performed by the HIPS 100 system and an electronic pill package with sensors and a processor that is in data communication with the HIPS 100. Process steps 1908, 1910, 1912, 1914, 1916, 1918, 1924, 1926, 1930 and 1938 are performed at the electronic pill package.

Based on the data gathered as described above, the HIPS 100 accesses the mendicant data 111 stored in the user account 110. The user may choose a guidance preference in 1902 for loading the pill package, e.g., by voice and/or text 1904. Thereafter, a multi-step process 1906 for loading the pill package is provided. In the example of FIG. 19, a five step process is shown, but more or fewer steps can be used.

After the user has indicated they are complete, the pill package checks for an unlocked compartment at 1908. If one is detected, then at 1910 the user is alerted, and at 1912, the user corrects the problem with the pill package. At 1914 the pill package signals the pill package is loaded and ready. In some implementations, the pill package may sense if pills are present, e.g., by weight. The pill package determines if all compartments with like dosages have like weights at 1916. If not, the HIPS 100 (or the pill package) notifies the user of the error, and provides guidance at 1920 for correction. At 1922, the corrections are made, and the compartment is ready.

At 1924, a compartment is opened and pills are removed. At 1926, the device determines if the compartment is empty. If not, the user is notified at 1930, and the user either empties the compartment or provides and explanation as to why some or all pills were not removed. The process proceeds to 1928, in which the HIPS 100 validates the pills are being taken in accordance with the dosing schedule. The process 1928 can be a rule based process or a machine learned process to generate the desired output for the set of inputs. For example, for a given dosing schedule, the process 1928 determines whether the corresponding pills are taken within a threshold time period of the scheduled time.

At 1932, the HIPS 100 determines if expectations are matched with the pill usage. If so, the date and time of the usage is recorded at 1936. If not, the user is advised to return the pills, continue, or provide an explanation. Finally, if pills are returned at 1938, the pill package returns to 1914 and the process flow resumes.

In some implementations, the HIPS 100 determines medications the user is taking and a schedule for each medication, and then determines a periodic dosing schedule for each medication. The dosing schedule indicates, for each medication, a time to administer the medication. The determining includes, for each medication, accessing medication data describing dosing requirements and constraints, medication interactions with other medications, and side effects of the medications. Dosing requirements can be, e.g., take with food or milk, and constraints can be, e.g., do not take while driving, etc. The HIPS 100 then determines, from the health data 110 of the user, an activity pattern data of the user that specifies user activities over multiple periodic dosing periods. For example, if the user often does not each breakfast in the morning and this is reflected in the user's health data 110, the dosing schedule for a pill to be taken with food may be shifted to begin at lunch.

Based on the medication data and the activity pattern data of the user, the HIPS 100 determines a periodic dosing schedule that specifies, for a time period and for each medication, a time to administer the medication. The HIPS 100 then sends to a user device of a user for display to the user the periodic dosing schedule. The user device can be a mobile device, a computer, or an electronic pill package with a display.

The HIPS 100 then monitors the dosing schedule for the user. This includes at each time to administer a medication, sending, to the user device, a notification to the user to administer the medication. For example, the user may receive a text message to take certain pills. In the case of the electronic pill pack, the data may cause the electronic pill package to generate one of an audible of visual notification for the user. The audible or visual notification can specify a specific compartment of the electronic pill package that stores a particular medication to be administered.

### Processing User Provided Data and Data Collection

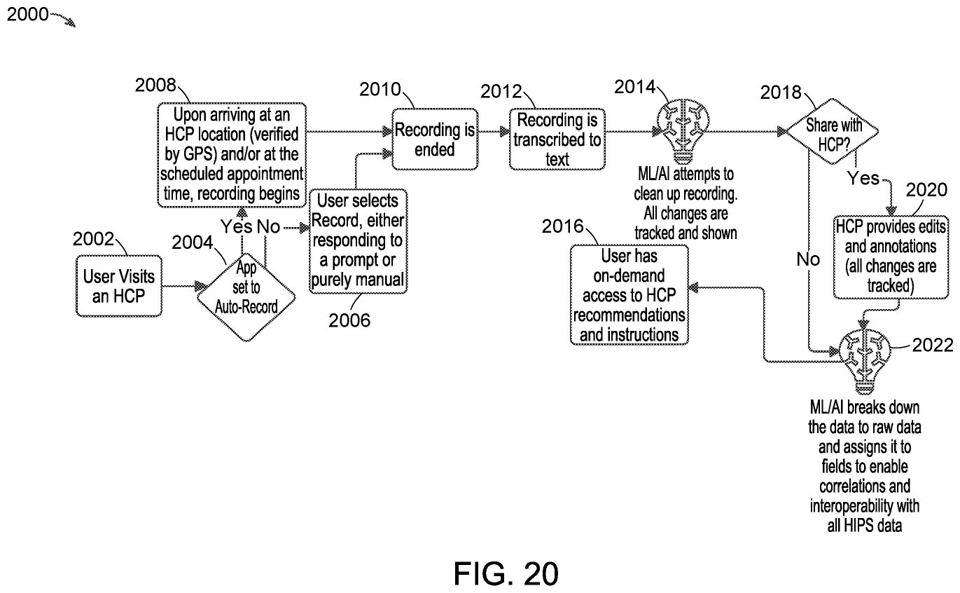

FIG. 20 is a system flow diagram 2000 of record process for processing user-provided records. The process allows for users to provide their own records in multiple different formats and to process the records and store the processed data in a standardized form for the HIPS 100.

Members, as patients, record HCP visits that are transcribed and converted to meaningful data and insights. While products exist for HCPs to transcribe their conversations with patients, this empowers members to provide their own observations and retain that data as part of their own records.

The recording can be done manually, e.g., after or during the visit the patient write their own notes, such as by use of an application linked to the HIPS 100, or simply in a text document for later upload. Another option is auto-recordation, e.g., by use a mobile device or a wearable. In some implementations, the wearable can double as a health monitoring device, e.g., a smart watch.

The wearable is set to record. The user may manually record, or, alternatively, the wearable may set to record at the appointment time. In the latter situation. The HIPS 100 processes that recorded data and determines time events for the visit. For example, by aggregating time events for certain HCPs, the HIPS 100 system can determine average waiting room times for each HCP. The extra time recorded in the recording in the waiting room may be removed from the file for file storage efficiency after the time spent in the waiting room is determined. A similar time metric can be evaluated after the user is brought in to see the health care professional. Conversations with the health care professionals are recorded and transcribed, and then, based on a reconciliation process (e.g., by time, HCP identity, etc.), are paired with the HCP records received from the HCP. In this way the user not only has the HCP records, but also has a complete transcription of the visit, and, optionally, any notes the user may provide.

At 2002, the user visits the HCP. At 2004, the device is activated by either a manual record operation or an automatic record operation. A manual record operation 2006 occurs when the user manually initiates recording using the application. An automatic record operation 2008 occurs when the user arrives at the HCP location and or at the scheduled time of the appointment. At 2010, the recording is enabled, and at 2012, the recording is transcribed to text. The transcription may occur in real time, or may occur after the recording is complete. The user may also add their own observations after the recording is complete, or while the recording is going on, by text.

At 2014, the data is uploaded to the HIPS 100, and processed using speech-to-text processing. Additionally, event times and durations may be determined, such as arrival time, waiting time, etc.

At 2018, the user has the option to share the recorded information with the HCP. If so, the HCP may choose to provide their own edits and changes in 2020, subject to tracking. At 2022, the HIPS 100 performs data conversion to the standardized HIPS 100 format and performs additional data analysis, such as waiting times, keyword flagging, etc. Non-specific user data may be aggregated for the HCP, e.g., wait times for multiple users, while specific user data (text and notes) are stored privately within the user account 110.

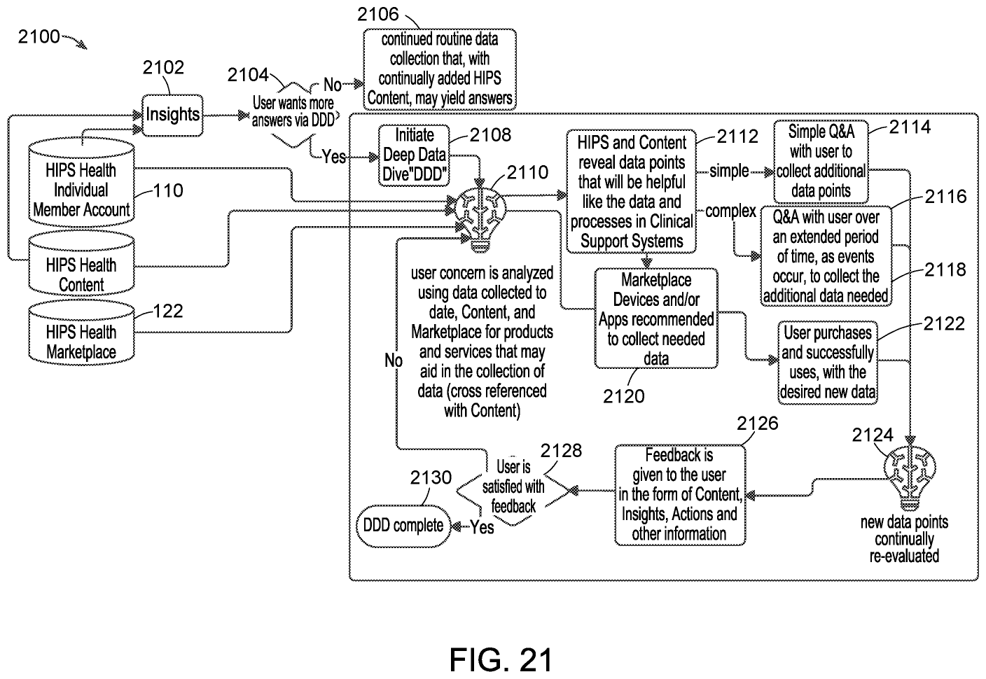

FIG. 21 is a system flow diagram 2100 of data collection and processing. The HIPS 100 collects data from a variety of sources, as described above. However, there may be situations when some data are underserved, e.g., when a member has a troubling new symptom and is struggling to determine the cause. When this occurs, the HIPS 100 can initiate a deep data dive (DDD). The deep data dive is used to interrogate something of concern or interest, and during the deep data dive, the member will expend much more time collecting minuscule data in a select area over a select period of time.

During a deep data dive mode, the HIPS 100 system inquires about additional data during data collection. For example, during a normal blood pressure collection, the HIPS 100 may only require the reading. But during a deep data dive, the HIPS 100 may generate a number of additional data requests to provide more context. These additional data requests may be machine learned or may be pre-associated with predefined metrics, i.e., the HIPS 100 can use an ML/Au/tool-powered process to ask relevant questions, or, alternatively, access a question rule set that is predefined. For example, during a deep data dive for blood pressure, the HIPS 100 may issue questions such as “How are you sitting? Where is your arm? What did you eat and when? Exactly how much did you exercise and when? Are you under increased stress for any reason?”

The HIPS 100 can also provide advice during a deep data dive based on the questions. For example, the questions can be turned into suggestions. In the case of the blood pressure reading, the user may inquire why a particular reading is high. The HIPS 100 may respond with “Alake sure you are sitting during the reading and have your arm resting on the table. Make sure to avoid foods high in sodium and caffeine before taking your reading. Wait several hours after strenuous exercise before taking your reading. If you are under increased stress for any reason, your readings may be elevated.”

Turning to FIG. 21, the user may receive insights 2102 as part of a general inquiry, or when uploading data. At 2104, the HIPS 100 or the user my initiate a deep data dive. The deep data dive may be initiated by the user manually, or may be initiated by the HIPS 100 based on the nature of the user's inquiry. For example, using semantic processing, the HIPS 100 may determine that the user's inquire may involve feedback from the user and additional information.

If a deep data dive is not initiated, then the HIPS 100 continues with a routine process at 2106. Conversely, the deep data dive is initiated at 2108, and at 2110, the HIPS 100 evaluates the user's concerns and inquires. The process 2110 can be any appropriate search and information retrieval process that can process user query inputs and other input to generate the recommendations. The recommendations may be for marketplace items, or for additional data to be collected for the user. Other types of health-related recommendations can also be generated and provided.

As shown in FIG. 21, a variety of data sources can be accessed. The evaluation may be based on machine learning or based on a predefined rule set. At 2112, the HIPS 100 determine data that may be of assistance to the user, and based on the complexity level, either issues a simple set of questions (e.g., one or more questions that are not dependent on answers), or a complex set of questions (e.g., multiple questions, where follow on questions may depend on previous answers).

In some implementations, the HIPS 100 recommends apps and or devices at 2120 that may be needed to gather data. These apps and devices can be linked to the HIPS marketplace, described below, or otherwise generally available. The user may then obtain the apps or devices at 2122.

At 2124 the new data, either answers to questions, or app and device data provided during use, are evaluated and then feedback is provided to the user at 2126. If the user is satisfied at 2128, then the deep data dive is complete at 2130. Otherwise, the process returns to 2110. Evaluation can be explicit or implicit. For example, the process 2128 may explicitly inquire of the usefulness of the data and based on the user response evaluate the data; or the process 2128 may implicitly evaluate that data based on users' actions or inactions taken after being presented with the data.

As can be appreciated, the deep data dive may be conducted during a single session by questions and answers, or may be conducted over an extended time, such as days or even weeks. In the case where apps and devices are recommended, the HIPS 100 realizes a technical improvement in data collection in that the HIPS 100 provides recommendations to data augmentation as part of the data collection process, which enables users and the HIPS 100 to continue analysis when the data would not otherwise be available.

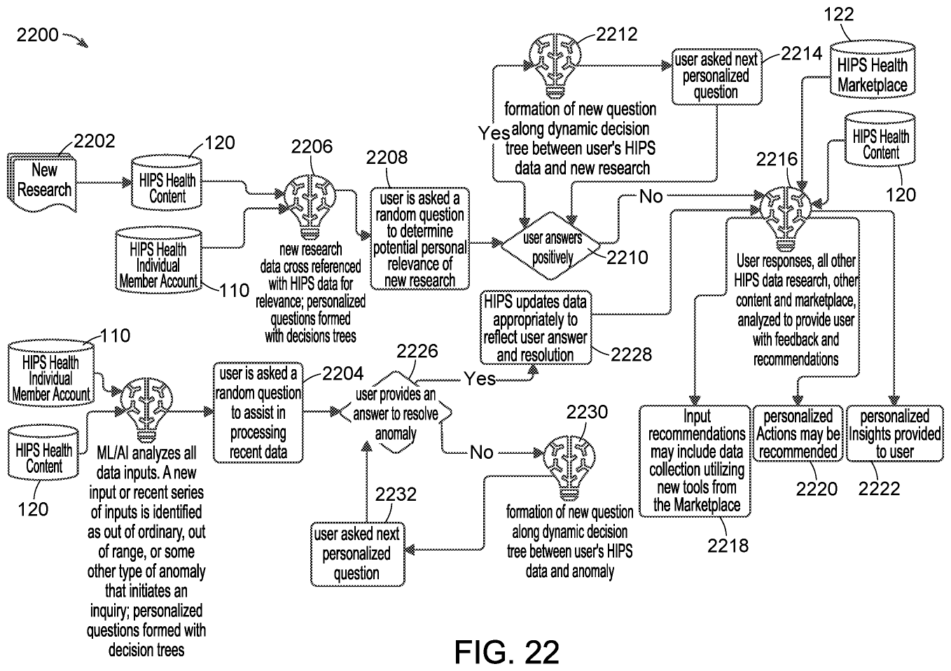

FIG. 22 is a system flow diagram 2200 of a question generation process for data collection. In some implementations, the HIPS 100 issues questions to the member. The questions can be triggered based on one or more events, such as developing research, compliance monitoring, illness, and the like. The HIPS 100 uses a predefined process or machine learning to determine when and what questions to ask.

As shown in FIG. 22, the question process may begin by either a new research trigger 2202 or in response to processing a recent data trigger 2204 resulting from the HIPS 100 determining that an input is anomalous, out of range, etc.

For new research, the HIPS 100 determines at 2206 relevant questions from a decision tree based on cross referencing the research data with the health data of the HIPS 100. The process 2206 can be a rule based process or a machine learned process to generate the desired output for the set of inputs. At 2208, the HIPS 100 poses the “random” question to the user. The question appears “random” in that it may be presented without context as to why it is being asked to avoid introducing bias into the answer, or simply random, with explanation, but beyond the user's current or recent focus.

At 2210 the HIPS 100 determines if the question answer is positive or negative. Positive means the answer is such that the HIPS 100 formulates another question based on the user response. Negative means the answer is such that the HIPS 100 has no more questions to ask, or the user refuses to answer.

At 2212, in response to a positive user answer, an additional question is formulated and then asked at 2214. The process 2212 can be a rule based process or a machine learned process to generate the desired output for the set of inputs. When the answer is finally negative, then at 2216 the HIPS 100 analyzes the answers and may provide recommendations. The process 2216 can be a rule based process or a machine learned process to generate the desired output for the set of inputs. The recommendations may include additional data collection using marketplace tools (2218), personalized actions (2220) and personalized insights (2224).

Returning to 2204 trigger, the HIPS 100 determines if the user provides an answer to resolve the anomaly at 2226. If so, the data is updated at 2228 and the process continues at 2216. If not, then at 2230 a new question is framed. The process 2230 can be a rule based process or a machine learned process to generate the desired output for the set of inputs. The question is based on a decision tree based on cross referencing the research and other data, such as comparison of the user's data to other users' data for anomaly detection, with the health data of the HIPS 100, The user is then asked the question at 2232, and the process continues at 2226,

### Insights

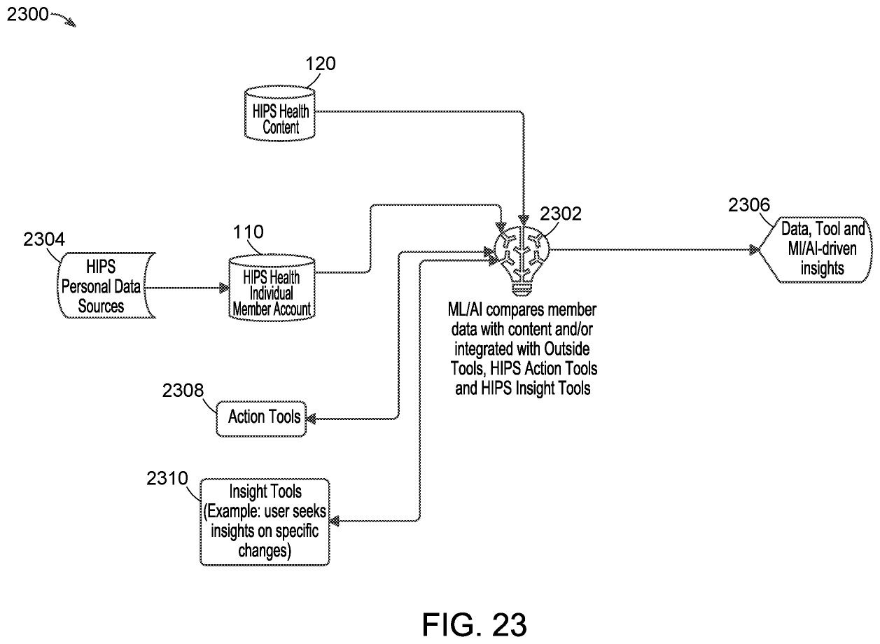

FIG. 23 is a system flow diagram 2300 of a data insight process. The HIPS 100 provides personalized insights to the user. Insights are guidance, data discovery, correlative findings, and educational information that are determined by the HIPS 100 to be of value to the user. An insight may be initiated by member inquiries or recent inputs, be part of a specific insight or action tool, or be automatically generated and presented to the member as an alert, or a general listing of observed insights that are continually evaluated and presented. Member and third party feedback on the validity and importance of the insights are continually integrated into the HIPS 100 system to refine and improve the future insights.

As shown in FIGS. 23, an insight process 2302 receives data from health content 120, member account data 110 that stores data from sources 2304, from action tools 2308, and from insight tools 2310. The data are processed to provide tools and insights 2306 to the user. The process 2302 can be a rule based process or a machine learned process to generate the desired output for the set of inputs.

Insights include single variable insights, multi-variable insights, and complex insights. Direct insights are drawn from a member's health data, whether single-variable recent changes, single-variable longitudinal changes, or complex multi-variable changes over the short-term or long-term. The insights may simply compare the member's data, and/or utilize comparisons of the member's data to standards, sourced from IPS content 120, account information 110, and other data sources 2302. Complex insights are similar to multi-variable insights, but take into account user personalization as described with reference to FIG. 8 above and filter accordingly.

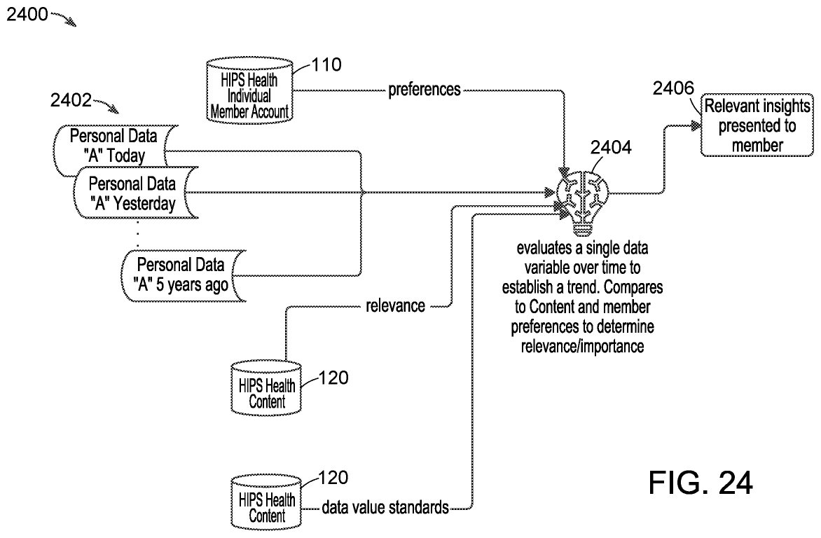

FIG. 24 is a system flow diagram 2400 of a single variable insight process. A single-variable longitudinal insight is an insight comparing only to the member's health data from the users account 110 and personal data 2402. An insight process 2404 evaluates a single variable over time to identify a trend, and it is presented as a relevant insight 2406. An example of such an insight is “You've been gaining weight steadily for the past 5 years,” or “Your cholesterol value taken yesterday is above the normal range.”

The process 2404 can be a rule based process or a machine learned process to generate the desired output for the set of inputs.

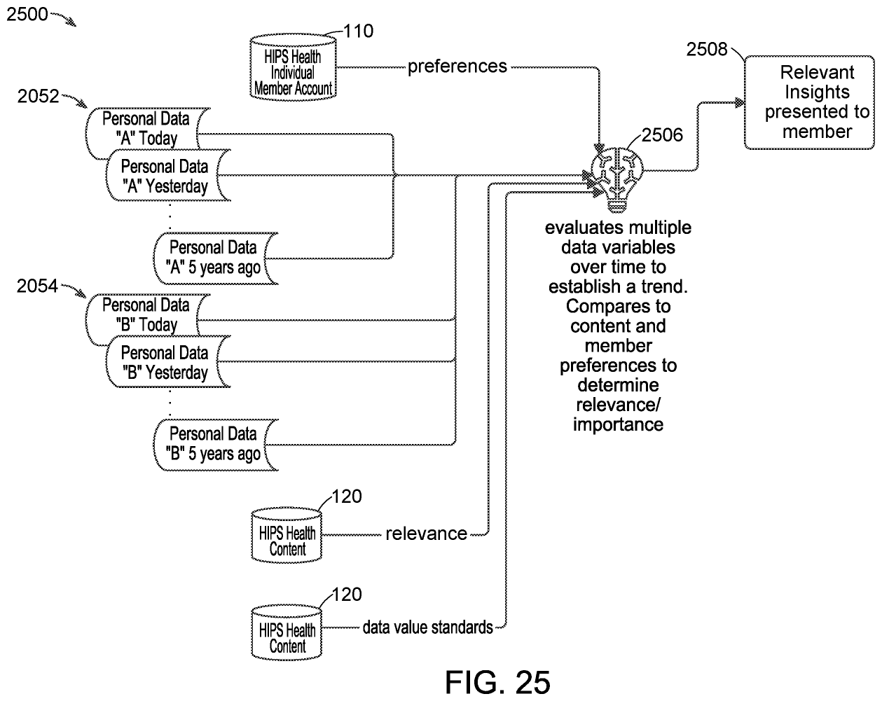

FIG. 25 is a system flow diagram 2500 of a multi-variable insight process. A multi-variable longitudinal insight can compare a user's data over the user's other data over time to identify an insight. The insight may also involve identifying potential causation, based on not only the user's account 110 and data 2502, but also comparing to another of the user's data, data 2504, to develop causation models. Causation, as used herein, may be inferred from a high correlation between facts, and a potential cause may be identified as a based on a correlation value achieving a threshold, for example. The insight process 2506 evaluates these variables to identify the insights and present the insights to the user at 2506. Examples of such complex insights may be “Your weight gain correlates to a reduction in exercise,” or “Your weight gain correlates to taking allergy medicines.”

The process 2506 can be a rule based process or a machine learned process to generate the desired output for the set of inputs.

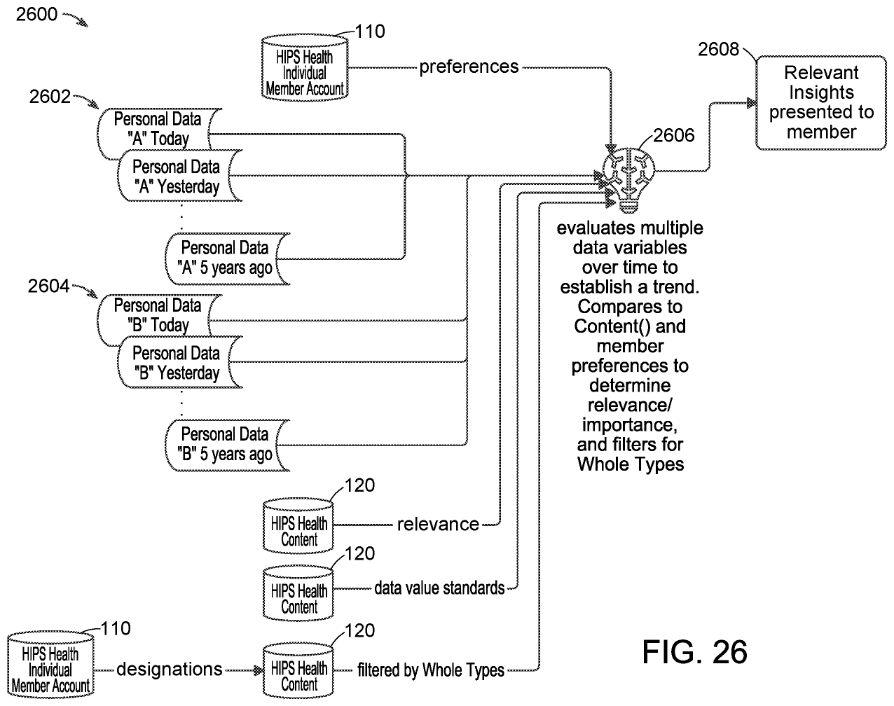

FIG. 26 is a system flow diagram 2600 of another multi-variable insight process. The process of FIG. 26 is similar to the process of FIG. 25, but the insight process 2606 takes into account user personalization when identifying and presenting insights 2608. For example, a user may have identified an “Urban Millennial Mother” as a personalization persona. The persona may be used to provide personal-specific recommendations. For example, a persona specific doctor recommendation may be “Because you have identified as an Urban Millennial Mother, Dr. Quasimodo is recommended as a top pick for your current needs, based on other Urban Millennial Mothers.”

The process 2606 can be a rule based process or a machine learned process to generate the desired output for the set of inputs.

### Obligation and Benefits Distilleries

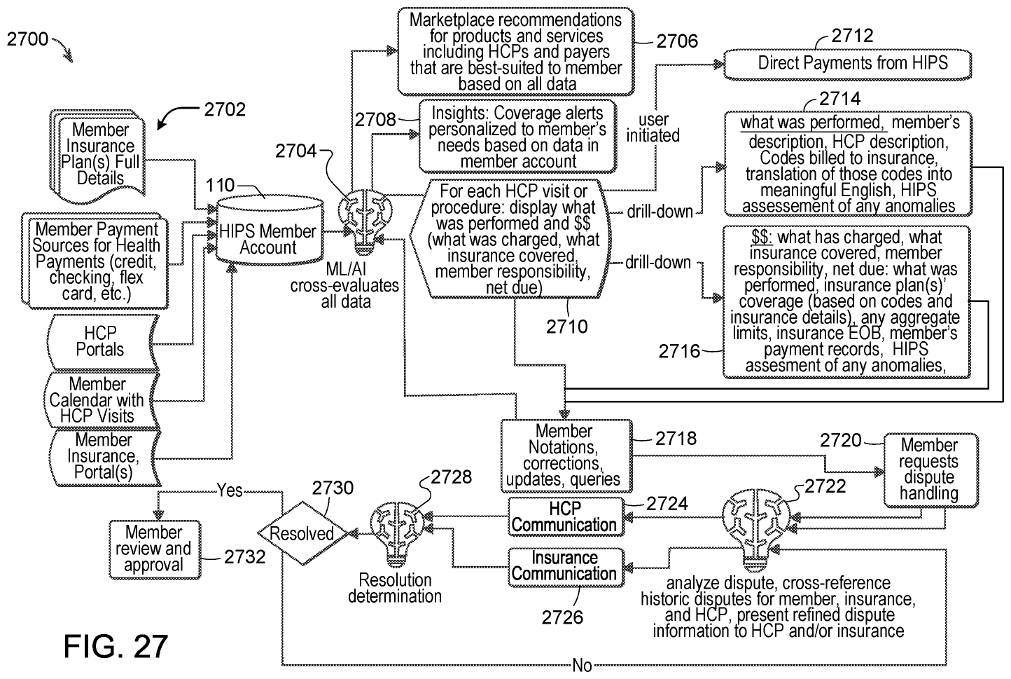

FIG. 27 is a system flow diagram 2700 of a financial distillery process. The HIPS 100 intakes member obligations, insurance plans, payment plans, and the like and provides the user summaries of the plans, payments, and services, with drill-down capabilities. For example, a user may be presented with summaries and drill downs directed to: 1) what the user has paid to whom and what is owed; whether the doctor bills match the explanation of benefits (EOB) statements; whether the user paid more than the EOB; coverage and non-coverage awareness; coverage recommendations, etc.

As illustrated in FIG. 27, the HIPS user account 110 stores data 2702 relating to the user's plans, payments, HCP portals, visits, insurance portals, and other data sources that are related to the user's obligations and benefits.

The HIPS 100, using a machine learning process or rule based process 2704, or, alternatively, an administrator, or a combination thereof, cross evaluates the data 2702 and generates marketplace recommendations 2706, coverage insights 2708, and obligation and payment summaries 2710.

The marketplace recommendations 2706 may identify productions and services best suited for the member based on the data 2702. The coverage insights 2708 identify coverage gaps or redundancies based on the data 2702. The payment summaries describe for each visit the procedures and examinations and what has been charged, paid and by whom. If the user has agreed, a direct payment 2712 can be made through HIPS 100. Drill down 2714 provides more information as to what was performed, billing and insurance codes, anomalies, etc. Drill down 2716 provides more information as to what was covered, what was charged, what is due, insurance EOBs, etc.

At 2718, the member may provide notations, corrections, etc. The process then returns to 2704 or ends, depending on the member's request. At 2720, the member may provide a dispute request and at 2722 the HIPS 100 analyzes the dispute and relevant data and presents a proposed resolution. The process 2722 can be a rule based process or a machine learned process to generate the desired output for the set of inputs. For example, the user may specify that certain items should not have been billed, and the process may pass the request on to the HCP/Insurance provider, or, based on historical data, may inform the user that such item are the responsibility of the insured.

The proposed resolution is provided to the HCP at 2724 and/or the insurance company at 2726. At 2728, the HIPS 100 receives data from the HCP and/or insurance company, and determines if there is resolution (e.g., payment in full, release of obligation, refusal, etc.). The determination can be made by a data indicator (acceptance or rejection, for example). If at 2730 HIPS 100 determines there is resolution, then the proposed resolution is presented to the member for acceptance. Otherwise, the process returns to 2722.

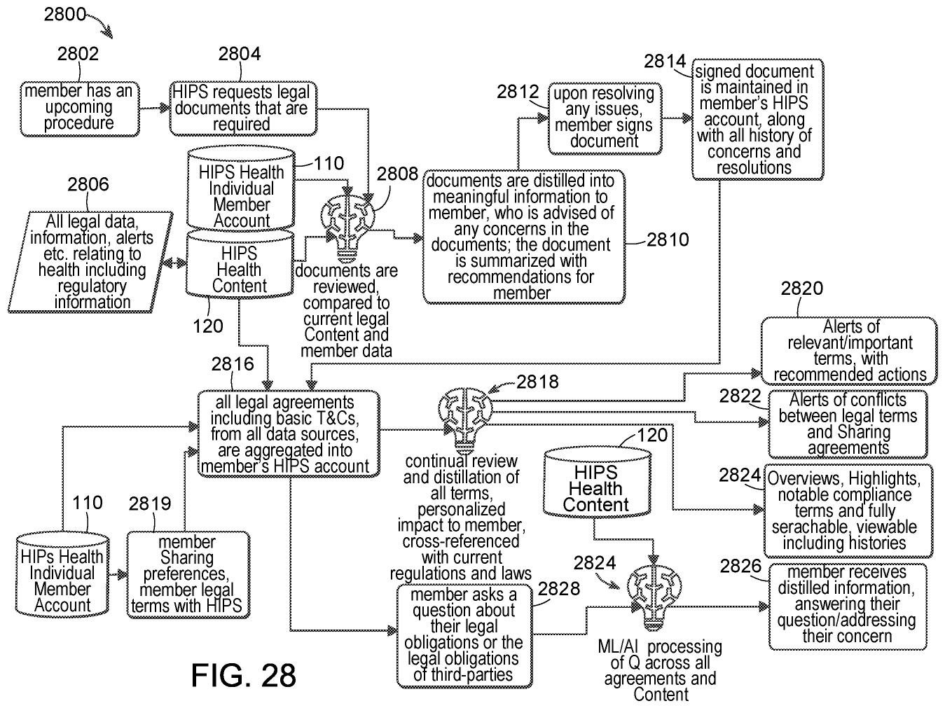

FIG. 28 is a system flow diagram 2800 of a legal distillery process. The HIPS 100 tracks and maps the legal obligations and permissions that are part of the member's relationships with personal data sources, HCPs, and beneficiaries. In some implementations, when inconsistencies between the obligations of the user and preferences are found, the user is notified. In other implementations, users are provided summaries of various documents prior to execution of the documents. For example, patients are often asked to sign a document for which there is no opportunity to completely read and comprehend. The HIPS 100 provides summaries that standardize and simplify these requests.

All legal forms a member has signed electronically and on paper (when member or designee scans) are maintained for reference. Dynamic agreements, such as terms and conditions in many apps that are subject to change without notice, are automatically monitored such that updates to terms are reviewed by HIPS and relevant information and changes are highlighted for the member.

Summaries are generated when a user has an upcoming procedure or appointment 2802. The HIPS 100 requests the legal documents that are required. They can be stored on the HIPS 100 if the HCP is also using the HIPS 100, or they can be provided electronically. Then the HIPS 100 implements a summary process 2808, which may also include accessing HIP health content 120 and other legal data 2806. The summary process 2808 can be machined learned to identify document type, e.g., HIPAA related documents, payment related documents, etc. The documents are distilled and are presented to the user at 2810. For example, a document, based on keywords, may be identified as a HIPAA release form. The distillation may state “This document is a HIPAA release form. It allows the ICT to share protected health information with other individuals or organizations listed in the document. If you sign this, the HCP can provide your information to others.”

At 2812, should the member have no issues or resolve the issues (e.g., by asking the HCP representative about the document), the member executes the document, and the signed document is maintained in the members account 110. This information is available to process 2818 for later processing as well.

Another function of the legal distillery is recommendation and alert processing. The process 2816 aggregates the legal documents on file for the user along with health content 120, and sharing preferences 2819 of the account data 110. The process 2818 then accesses this aggregated data and, using a machine learning process, or a rule based process, or, alternatively, an administrator, or a combination thereof, generates recommendations and alerts. Examples include alerts 2820 to review important terms and recommended actions; alerts between conflicts between legal sharing obligations (e.g., sharing data for a drug trial) and sharing preferences and overviews and highlights 2824 regarding compliance terms.

Distilled information can also be produced in response to a member question. For example, at 2828, a member may inquire about a particular legal obligation, e.g., “What is this HIPAA release for?” The process 2824 may then generate the summary of distilled information 2826 to answer the query, in a manner similar to the distillation process 2808.

### User Queries

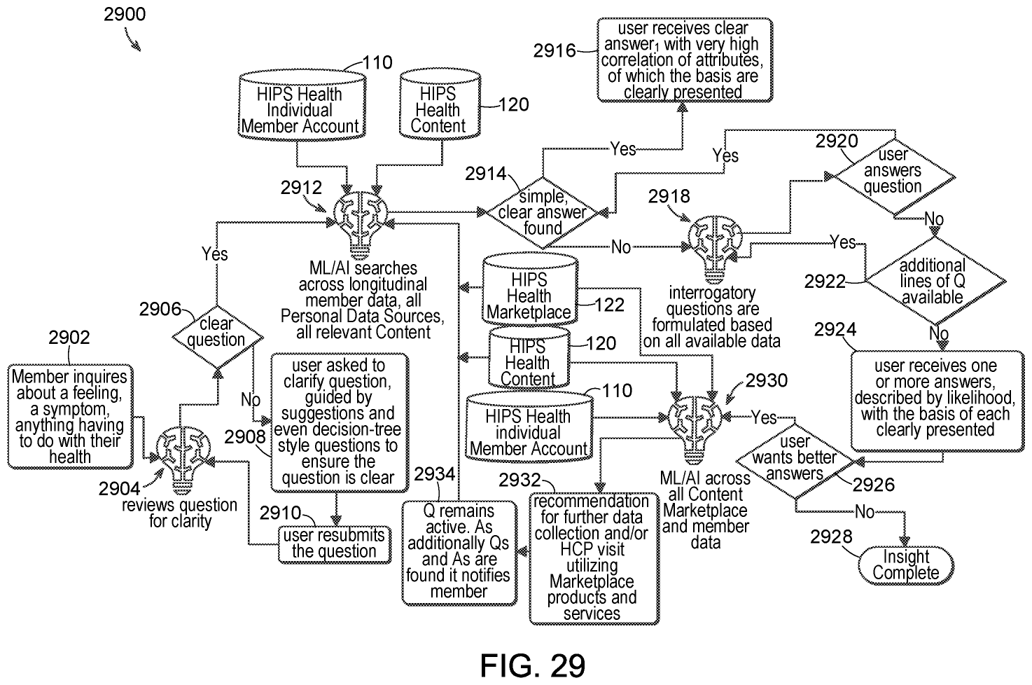

FIG. 29 is a system flow diagram 2900 of another query process. The HIPS 100 includes a query system that allows a user to seek answers to questions about their health. The HIPS 100 takes into account the user's personal information for generating an answer. For example, a user may ask “I've felt great since May 24. What factors may have contributed besides the one I can think of?” The HIPS 100 examines user account data 110 and all other data (e.g., health marketplace 122, health content 120, etc.). By processing the user's data specifying sleep, heart rate, exercise, vitamins, stress level, time at desk, food, etc., the HIPS 100 can, by use of machine learning, heuristics, or a rule based system, determine correlations between the user's condition expressed in the query and the user data.

The process begins at 2902, when a user inputs a query. The query is then processed at 2904 to determine if it is well formed, e.g., by use of semantic and syntactic language models. At 2906, the system determines if the query is clear. If not, the user asked to clarify at 2908, and the resubmitted query 2910 is again processed.

Once clear, the HIPS 100 processes the query at 2912 to determine one or more answers. The process 2912 can be a rule based process or a machine learned process, or other appropriate search algorithm.

At 2914, the HIPS 100 determines if an answer is found. The determination can be based on a relevance scoring model, for example. If the answer is found, it is presented at 2916. If not, the system at 2918 requests more information from the user, e.g., “You mentioned you have sinus pressure. Do you have a fever accompanying this condition?” The process 2918 can be a rule based process or a machine learned process to generate the additional requests based on prior requests and responses.

If at 2920 the user answers and no more questions are required, the process returns to 2914. Conversely, additional questions can be asked at 2922, and answer can then be provided at 2924. If the user is satisfied at 2926, the insight is complete. Otherwise, at 2930, the HIPS 100 accesses additional data, such as the marketplace 122, for example, and provides additional answers or recommendations 2932. The process 2930 can be a rule based process or a machine learned process, or other appropriate search algorithm.

Finally, because the user has not expressed satisfaction with the answer, the HIPS 100 keeps the question active at 2934, and periodically re-evaluates to determine if new answers are available. If so, the new answers are presented to the user.

### Content Curation and Marketplace

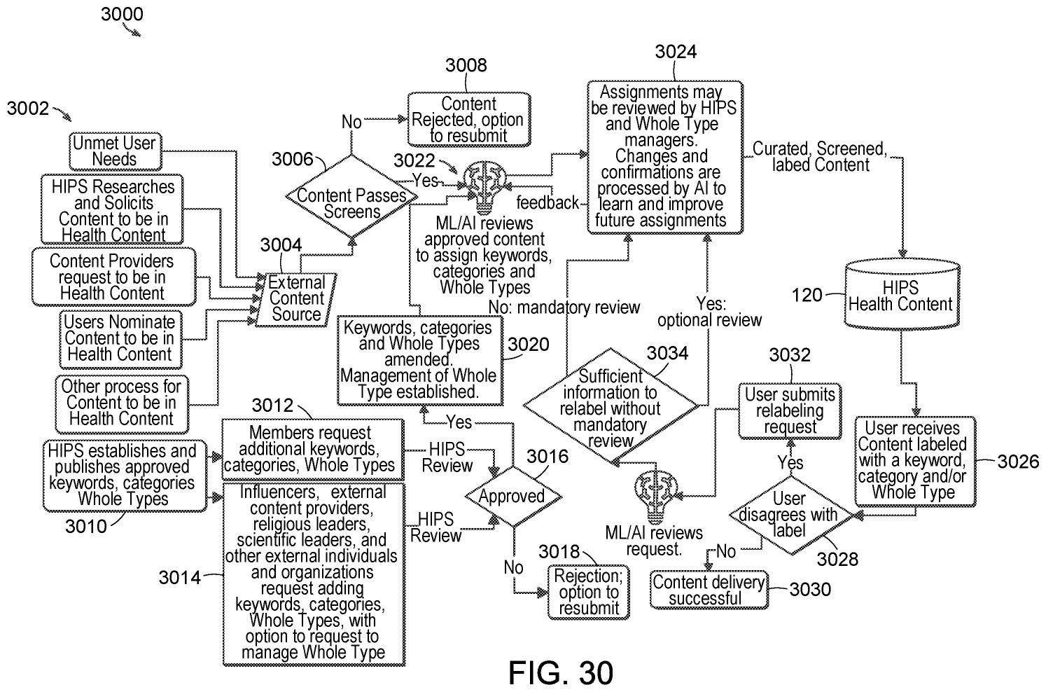

FIG. 30 is a system flow diagram 3000 of a content curation process. The health content 120 includes health-related content that is not necessarily specific to particular users but is accessed by the HIPS 100 with health data of the HIPS user account 110. Using these two data sources, the HIPS 100 provides personalized content that is relevant to the user's health. Examples include research that is relevant to the member, data regarding drug interactions, journal articles, etc.

The HIPS 100 can processes content and generate content tags and index the text of the documents by means of automated processes, such as keyword identification, term-frequency/inverse document frequency analysis, cross-reference weighting, machine learning/artificial intelligence, and the like. Additionally, curators can also manually review and tag content. Curators include employees and external participants. Their objective is to continually seek content and content resources/aggregators. The resulting corpus, with content tags and the indexed text, enables the capabilities described above, such as the insights, identifying search results to queries, and persona filtering.

In some implementations, during the content curation, if new content is determined to have information relevant or important to a member, the information can be pushed to the user by means of an alert, text, e-mail or some other manner. For example, assume a new side effect of a drug is identified, there is a recall of a particular nutritional supplement, etc. As this information is processed by intake, users of the drug or the nutritional supplement are notified of the new information. In some implementations, the notification includes a response option to notifying one or more HCPs if the user is experiencing the symptoms listed in the notification. If the users are experiencing adverse effects listed, selection of the response option will cause the HIPS 100 to send a message to the HCP(s) detailing that the user is experience the symptoms.

The creation and maintenance of health content 120 begins at 3002, where content is identified in a variety of ways. For example, users may express a need for certain content, in which case administrators may search for or develop the content, or solicit the content; content providers may submit content to the HIPS 100; users may nominate content for inclusion, and other processes, such as feeds from journals, web crawling, and the like.

At 3004, the data are received from external content sources, and at 3006, the HIPS 100 determines if the data passes a screen. The screening may involve spam review, reputation review, etc. If the screen is not passed, at 3008 it is rejected, and if there was a submitter, they may be given the option to resubmit.

Also at 3010, the HIPS 100 publishes approved keywords, categories and types. Members may request additional categories and types at 3012, and external providers may also do the same at 3014. The HIPS 100 reviews the requests at 3016. If rejected, the submitters may be given the option to resubmit at 3018. If approved, the keywords, types and categories are amended at 3020.

At 3022, the submitted content and the keywords, types and categories are processed by an indexing process to index the data and assign keywords, categories and types. A variety of indexing processes can be used, as described above. At 3024, the results may be optionally reviewed by administrators. Feedback can be used to tune the process of 3022. If approved, the content and resulting metadata (indexing, keywords, categories and types) are persisted to the health content 120.

Users may also aid in the curation of content. At 3026, a user may receive content and its corresponding metadata. The user may indicate whether the user agrees or disagrees with the metadata. For example, the user can initiate at 3028 a review process if the user disagrees, or the user can be requested, at random, to state whether the classifications, etc., of the data are accurate.

If the user agrees, then at 3030 the content is deemed correctly processed. If the user disagrees, the user may submit a labelling correction request at 3032, and an automated process reviews the request at 3034 for completeness. If sufficiently complete, then the process returns to step 3024. Otherwise, the request may be manually reviewed, adjusted, and then the process returns to 3024.

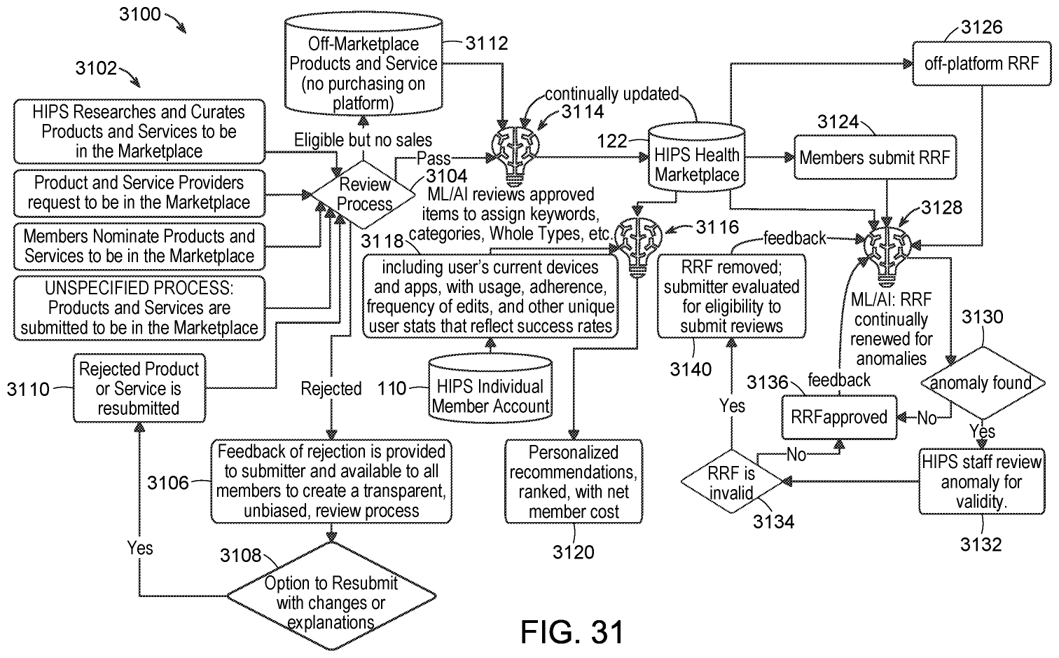

FIG. 31 is a system flow diagram 3100 of a health marketplace process. The HIPS 100 implements a “marketplace” for devices, apps, HCPs, and any other products or services designed to help people manage their health, that impact their health, or that are utilized by HIPS 100 tools and interoperable third-party tools. The HIPS 100 provides recommendations and links for recommendations, when appropriate. Recommendations can be provided through insights, actions, by use of tools, etc. Recommendations may be prioritized based on reviews, ratings, and relevance to the member's data and preferences. The reviews and ratings are provided by a reviews, ratings and feedback (RRF) system. Data describing the products and services can also be processed in a manner content is processed as described in FIG. 30 so that metadata for indexing, categorization and type processing is generated.

The HIPS 100 also includes tools for the health marketplace 122 that facilitate finding and connecting with an HCP for the member, based on personalized selections incorporating the member's insurance, other personal preferences recorded within HIPS, member's HIPS data, current individual needs, etc.

Products and services can be proposed for inclusion into the marketplace in a variety of ways at 3102. For example, employees can propose goods for inclusion; the providers of the goods (products and/or services) can propose for inclusion; members can propose for inclusion; or any other process by which goods and services can be proposed.

At 3104, the proposals are reviewed, either by an automated process or by reviewers. If a proposal is rejected, then at 3106 feedback is provided. If an option to resubmit is provided at 3108, then the rejected submission can be revised and submitted again at 3110.

The review at 3104 may determine that the goods are eligible for the marketplace but are not available for sale or purchase through the marketplace 122. These are categorized as off-marketplace goods, as indicated by 3112. For example, some goods may have exclusive sales channels not available to the marketplace 122. The proposals are then reviewed at 3114, in a manner similar to the process of FIG. 31, to generate descriptive metadata for search, suggestions, and the like. They are then persisted to the marketplace 122.

The marketplace 122 is then accessed by a recommendation/search process 3116, which also takes into account user account data 110. The process 3116 can be a rule based process or a machine learned process, or other appropriate search algorithm. The marketplace relevant data 3118 may include the users of current devices, apps, insurance coverage, etc. From this data 3118 and the marketplace 122, the process 3116 can generate recommendations, suggestions, and search results for the user. The marketplace 122 can also provide recommendations to HCPs for goods and services that may be of use to the HCP in providing care to their patients, 3206 and 3208.

Reviews, ratings and feedback (RRF) data can be provided by members 3124 and also gathered from off-platform sources (e.g., a separate shopping service review, etc.). Reviews are then processed for anomalies at 3128. Anomalies include span reviews, targeted/inflating reviews, etc. The process 3128 can be a rule based process or a machine learned process trained to detect anomalies. In no anomaly is found at 3130, then the RRF data is approved at 3136 and can be used for rating the goods and generating updated RRF data. Otherwise, the RRF data can be rejected outright, or can be reviewed by reviewers at 3132. The human review may find the anomaly to be a false positive; if so, the RRF data is approved at 3132. Otherwise, the RRF data is removed, and the submitter may be evaluated as to whether the submitter can submit future reviews.

### HCP Product Suite

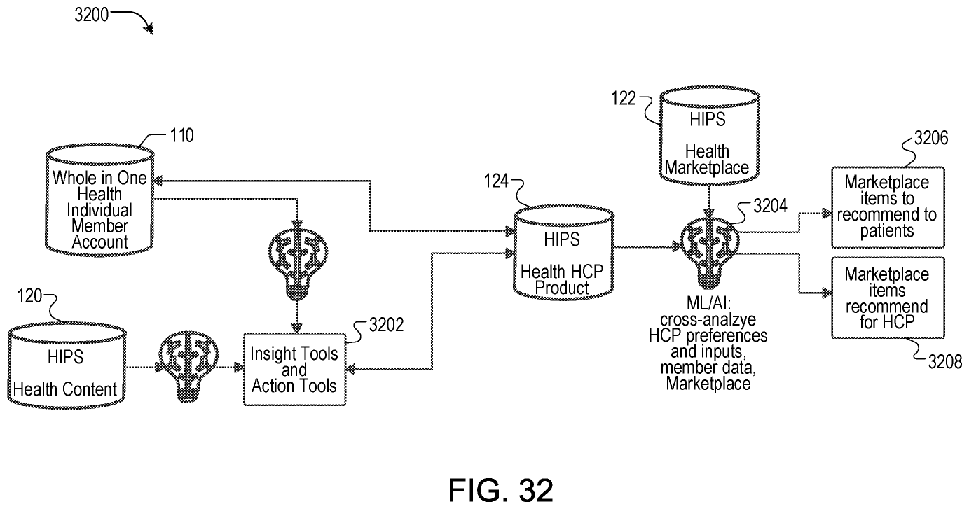

FIG. 32 is a system diagram 3200 of a Health Care Provider (HCP) product suite system. The HIPS 100 also includes HCP accounts through which HCPs can record and access data for their patients and provide services to their patients. Many of the resources, benefits and tools available to the users are also available to the HCPs.

Each HCP may purchase a HIPS EMR or develop and manage their own EMR systems, and each FMR system may thus have unique data schema. As described above, the particular EMR system used is not a barrier to entry to the HIPS 100, because the HIPS 100 includes technology that standardizes records in a standard HIPS 100 format.

As shown in FIG. 32, the general HCP product 124 provides HCPs access to various insight tools and action tools 3202 that are also available to users. Additional insight tools and action tools specific to HCPs can also be implemented. Likewise, a marketplace process 3204 allows HCPs access to the marketplace 122, through which marketplace items can be recommend to patients at 3206 and marketplace items can be recommended to the HCP at 3208. The process 3204 can be a rule based process or a machine learned process, or other appropriate search algorithm to provide recommendations as search results.

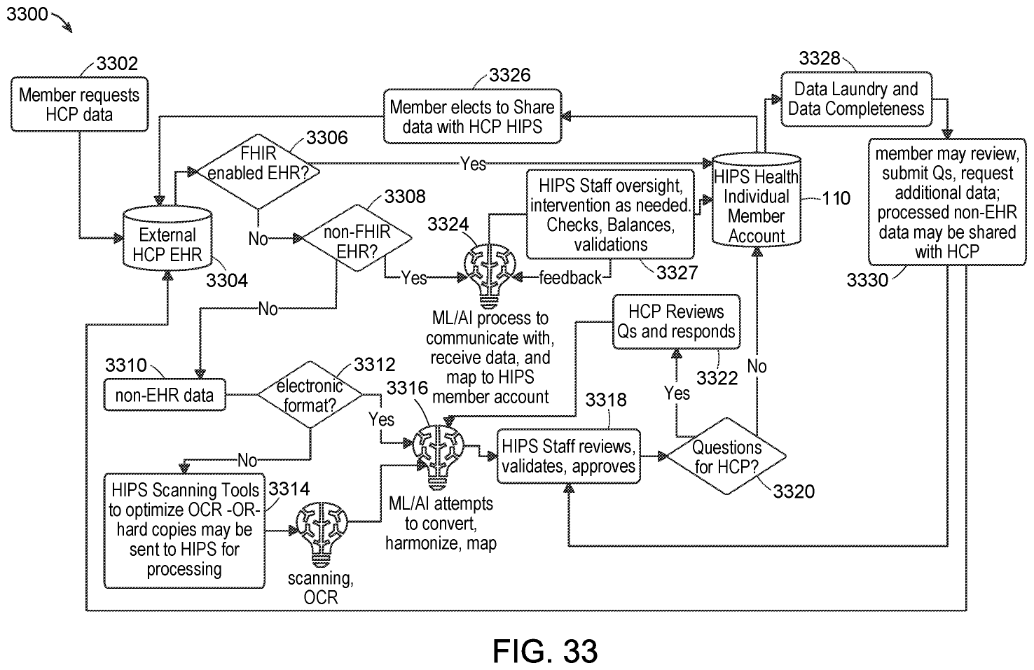

FIG. 33 is a system flow diagram 3300 of an HCP record management process. Some EHR systems will allow for relatively automatic data transmission, requiring no effort for the HCP, e.g., EHR systems that comply with FHIR. The HIPS 100 provides supplemental tools to streamline the data that is not in their current EHR. This may include ongoing data that is outside the scope of the EHR or is not recorded within the EHR for another reason. It also includes historic data that never made it into an EHR or that was in a previously used EHR and did not make the transition. In FIG. 33, the HCP utilizes its own EHR and data is accessed by the member though the HIPS 100, or, as shown in FIG. 33, through the HCP at 3304 (e.g., through an HCP portal).

The HCP EHRs 3304 can be provided for inclusion with the member account 110. If the records are FHIR compliant, as determined by 3306, then the records can be imported to the account 110. The HIPS 100 may also use FHIR compliant schemes, or, alternatively, may use a proprietary scheme for which the FHIR records are converted.

If the records are not FHIR compliant, then at 3308 the HIPS 100 determines if the data are electronic health records, or some other data. If the data are EHRs, the process 3324 converts the records, and the conversion is optionally checked at 3327 by staff. If approved, the data is persisted to the member account 110. Otherwise, adjustments and corrections are made.

If the data are not EHR, e.g., some other data format or data 3310, different steps may be taken. For example, if not in electronic format, as determined by 3312, the data may be scanned and optical character recognition (OCR) may be performed. The scanned data, or electronic data, is then processed by 3316 to convert and map the data. Any appropriate mapping and conversion process can be used. The output is then optionally reviewed by staff at 3318. If there are questions of the HCP at 3320, the HCP can review and respond at 3322 and the process returns to 3316. Otherwise, the data is associated with the user account 110.

Finally, the member may implement sharing at 3326, and share other data with the HCP. Additionally, the member may implement data cleaning at 3328 and the resulting records are reviewed and submitted at 3330. For example, at 3330, the member may review and submit questions, request additional data, and provide non-EHR data (e.g., scanned paper forms or other data). This may be optionally reviewed at 3318, and if there are questions of the HCP at 3320, the HCP can review and respond at 3322 and the process returns to 3316. Additionally or alternatively, the output of 3330, when appropriate, may go directly to the HCP via HCP EHRs 3304 and the process may return to 3306.

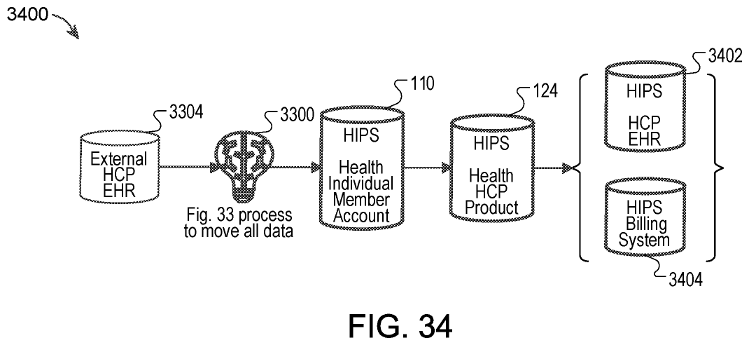

FIG. 34 is a system flow diagram 3400 of a HIPS HCP Electronic Health Records (EHR) process. In the process of FIG. 34, the HCP establishes an EHR within the HIPS 100. The records 3304 are processed at shown in FIG. 33 by the overall process 3300 and stored in the member's account 110. The HIPS HCP FHIR may duplicate, replace, or solely fill the need for their own EHR through the HIPS 100. These HCP records 3402 are then maintained in the HIPS 100. Optionally, billing for the HCP may also be managed by the HIPS 100 through a billing system 3404.

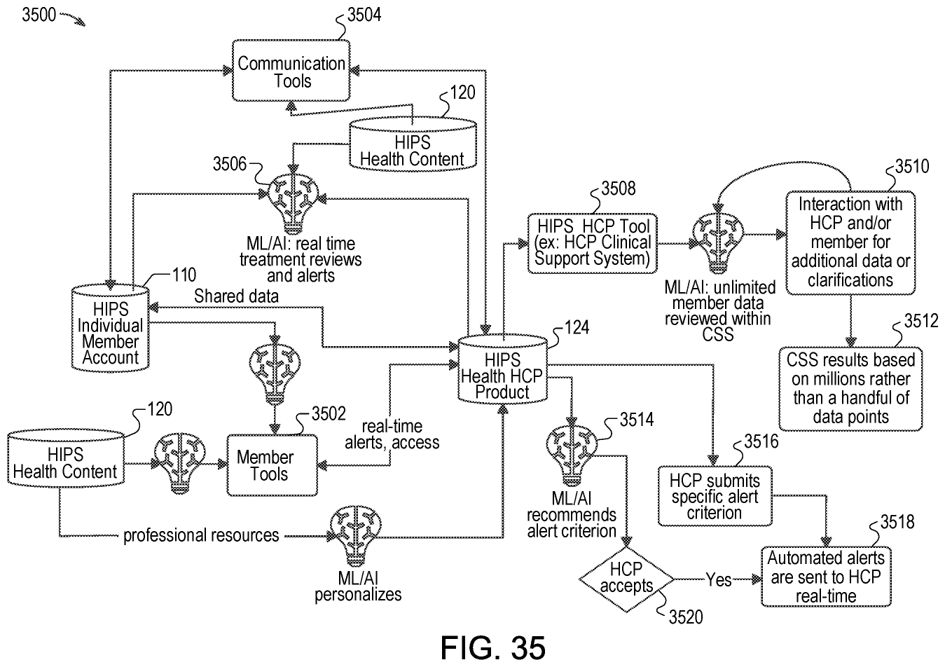

FIG. 35 is a system flow diagram 3500 of interactions between an HCP and member account. The flow diagram 3500 describes full interaction between the HIPS 100 and the HCP for a particular member account 110. In particular, the HCP may utilize not only member tools 3502 (insights, actions, etc.), but also professional HCP tools are integrated in HIPS 100. Such tools allow for specific HCP alert setting and enabling the HCP to incorporate the member's data directly into other tools such as a Clinical Support System (CSS).

The HCP's own capabilities are also augmented. For example, the HCP can receive immediate feedback if a treatment it recommends could cause issues that may not have been realized by the HCP without additional data stored HIPS 100 member account 110. The HCP also has access to communication tools 3504 between HIPS HCP EHR and member account 110 such as appointment making, inquiries, specific/push data sharing, etc.

Additionally, a HIPS 100 process 3506 can monitor the health content 120, the HCP product 124, and member account 110 and provide recommendations and alerts to the HCP in a manner similar to the way recommendations and alerts are provided to members.

Other tools include, for example, a CSS HCP tool 3508 that allows the HCP to provide data to a CSS (provided the member has agreed to share such data). The data being provided may be reviewed at 3510 by the HIPS 100 for adjustments before sending to the CSS 3512.

Another tool is an HCP alert system. Alerts for the HCP may be processed by an alert processor 3514. For example, if a user is due for an annual skin screening, but no appointment has been made, the HIPS 100 may alert the HCP 124 in addition to, or in lieu of, the member. At 3520, the HCP has the option to opt-in to such auto generated alerts, or reject them from being processed. The HCP may also set their own alerts in 3516 for future alerts 3518.

### Beneficiaries

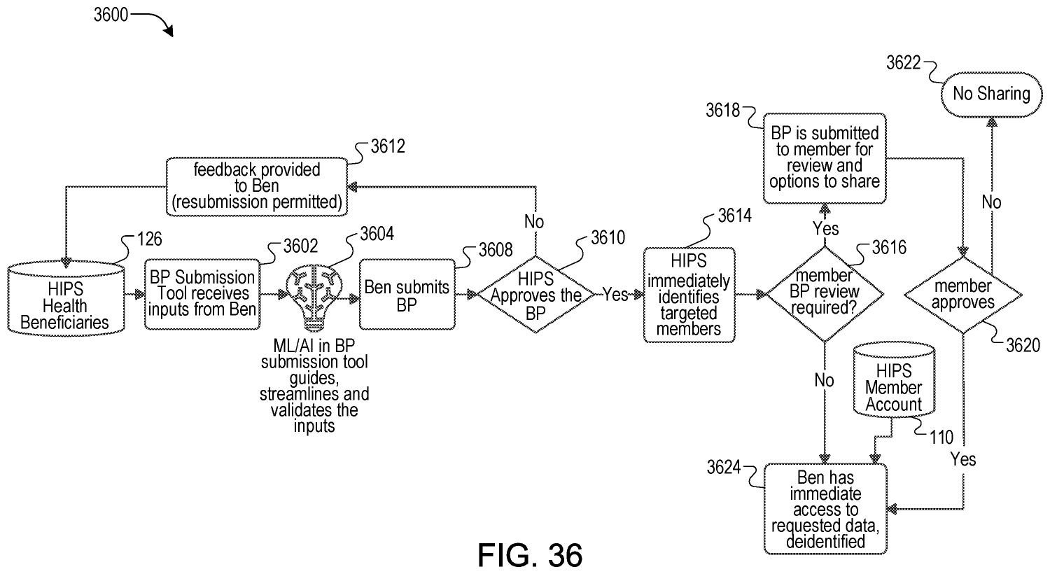

FIG. 36 is a system flow diagram 3600 of beneficiary proposal process. As used in this specification, a beneficiary is a third party that benefits from access to a member's data or providing the member's data to another party. For example, a beneficiary may be a third party conducting a clinical study; a third party that provides services for members; or any other party that derives gain, either monetary or otherwise, from use of the member data.

Beneficiaries submit applications to be includes as beneficiaries in the HIPS 100. Once approved, the beneficiaries may then engage with users for mutual benefit. Once enrolled, a beneficiary submits a beneficiary proposal, and, if approved, the proposal is then included in the HIPS 100 as an active beneficiary program. The proposal provides a detailed explanation for member data sharing, and explains the goals of using such data, how the data will be used. Extensive guidelines for the proposal, along with the preparation tool, are provided to all enrolled beneficiaries. Automated tools also enable automation and personalization of the preparation process to occur between the information systems of the beneficiaries and the HIPS 100.

Once a proposal is approved, access to member data can be immediate, typically upon member approval. The beneficiary may further refine the proposal to include the targeted members at any time, pending review and approval by HIPS 100. The detailed proposal is integrated into HIPS 100, enabling immediate identification of the targeted members. Targeted members may have a variety of share elections in place, ranging from “Share All, no Beneficiary Proposal Review Required,” where their data can be shared immediately, to “Share no data without my explicit consent”, where they are open to reviewing proposal, but must approve each data share, to many elections in between.

Upon reviewing, a member decides whether to share, and to what extent. The IPS 100 can deidentify (anonymize) all data regardless of the extent of sharing the member selects. In some cases, deidentification may not ensure complete anonymity (e.g. only several members participate), in which case the members are provided with this information to aid in their decision to share or not share.

A member may also specify blind or unblind requests. In a blind request, a beneficiary will submit a proposal to the member without any screening as to whether the member is in the target data acquisition group. Members may opt to be in this group but minimize any sharing of their data used in targeting a group, until they review the proposal.

In an unblind request, a beneficiary can search for specific criteria in member data among those members that allow their identification to be known. Through the IPS 100, the beneficiary submits a proposal to those that meet the criteria. This reduces requests that may not apply to the member.

The flow diagram 3600 is one example of a beneficiary proposal process. An approved beneficiary 126 provides a proposal by use of the submission tool 3602. The submission tool 3602 implements a submission process 3604 to assist the beneficiary in generating the submittal proposal 3608. The process 3604 can be a rule based process or a machine learned process. At 3610, the HIPS 100 either approves or disapproves the proposal. If disapproved, feedback is provided to the beneficiary 126 at 3612. Otherwise, if approved, the HIPS 100 can identify targeted users or users that meet selection criteria at 3614. The users are not disclosed to the beneficiary; instead, the member preferences are accessed to determine if the member requires review and acceptance. If so, the proposal is submitted to the member for review at 3618. If the member disapproves at 3620 then no sharing occurs at 3622. Otherwise, if the member approves, or does not require approval at 3616, the beneficiary has access to the data at 3624. The data may be anonymized.

### Illness Metering and Monitoring

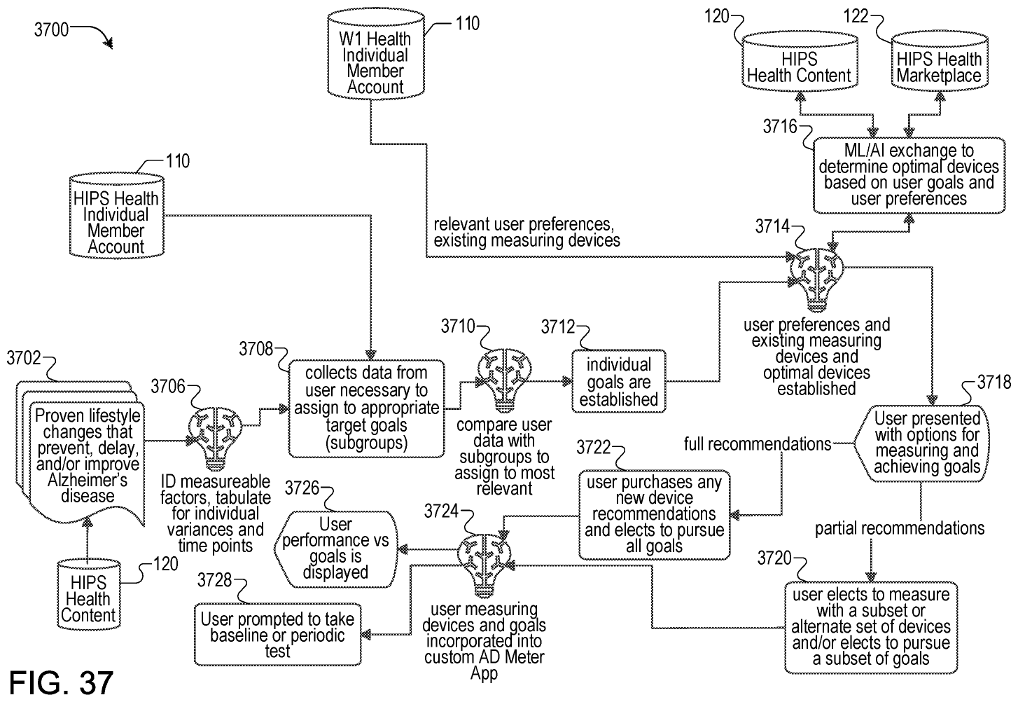

FIG. 37 is a system flow diagram 3700 of illness metering process. People may have illnesses that require monitoring and also require lifestyle changes to manage the symptoms. Additionally, there are diseases a person can offset or even prevent by implementing lifestyle changes and goals. However, there are several problems in doing so. First, if there are more than just a few goals and actions a user must take, the user cannot practically assess, recall, and calculate personal performance across these factors for a given day, let alone multiple days or extended periods. Thus, the seemingly simple collection of the data is challenging as memories rarely are accurate. Accordingly, data collection utilizing data collected in applications and devices is integrated into the system. Moreover, by use of the health marketplace, the HIPS 100 can identify and recommend tools and devices for the user that will assist the user in tracking the daily activities for illness metering and monitoring. In particular, the HIPS 100 not only monitors for user compliance for illness management for a given illness, but also provides the ability for the user to select instrumentation that achieves full monitoring and thus compliance.

In FIG. 37, the RIPS 100 accesses data that describes goals (activities, diet, exercise, etc.) for preventing, delaying, and improving a particular disease. From those findings, measurable factors for achieving those goals are identified, including any variance for age, demographics, sex, or other discernable reasons. Factors/requirements to achieve the goals are established over a period of time, such as daily, weekly, monthly and annually. User goals may vary by user's age, gender, etc. Once goals are set, the user is guided with options for measuring each goal, including recommended tools and devices. Once the measuring devices and/or methods are established, the member is measured and presented with feedback over the designated period of time to determine if they are achieving the goals. Thus, rather than recommending a fixed set of tools and devices for goal monitoring, the HIPS 100 updates recommendations based on the latest research, and can change goals and tool and device recommendations as needed. The user is thus presented with a dynamic system that monitors goals for illness management according to the latest and most up to date research.

In operation, the HIPS 100 receives, for each of one or more diseases, data that describes activities, diet, exercise, and other behavioral and activity changes that can offset, mitigate, or prevent onset of the disease. The data may be from the health content 120, or from some other source.

At 3706, the HIPS 100 processes the data to identify measurable factors that can be set as goals for users. The process 3706 can be a rule based process or a machine learned process, or other appropriate process that can associate factors with goal. For example, sleep, exercise, and diet goals can be ascertained and set for a particular disease. At 3708, the HIPS 100 collects data for a user from the user account 110 to assign the user to a group. Because goals may be different for differing groups, e.g., a first goal set for a first group that is female, a second goal set for a second group that is male, etc., the HIPS 100 compares, at 3710, the user data with various subgroups to assign the user to the most relevant group. An appropriate categorization algorithm can be used. Then, at 3712, individual goals are established for the user.

At 3714, the user's tools, devices and monitoring capabilities, determined from the user account 110, are compared to the goals, and an optimal set of tools and devices are established. If the user uses the recommended tools and devices, the reporting compliance for the goals is greatly increased. The tools and devices can be determined, for example, by a process 3716 that accesses the health content 120 and health marketplace 122. For example, one goal for a particular disease may be to ensure that blood sugar levels are strictly controlled. Accordingly, the process 3716 may rule out single instance glucose meters that require manual assays, and instead recommend only continuous monitoring glucose meters that are app enabled to report hourly readings back to the HIPS 100. The process 3716 can be a rule based process or a machine learned process.

Because for each set of goals there is a corresponding set of reporting requirements, each set of goals may result in a different set of monitoring tools and devices. By way of another example, a goal for another disease may be to ensure that glucose is monitored before going to bed. Thus, instead of a recommending only continuous monitoring glucose meters, the HIPS 100 may also recommend single instance glucose meters.

Moreover, goals may be different for the same disease among different users. For example, two users may desire to minimize their risk of diabetes. A first user may not be overweight, while a second user may be very overweight. Thus, the goals for the second user may include losing 35 pounds, and the exercise goals for the second user may be less, initially, than the exercise goals for the first user.

Moreover, goals are dynamically adjusted. As the user's health data changes, so may the goals. For example, once a user establishes an aerobic base, the user's recommendation for cardiovascular exercise time per week may be increased.

The user is then presented with the options for measuring and achieving goals at 3718. The presentation may be via a web portal, an application, or by some other process. The user may only take part of the recommendation at 3720, e.g., the user may already have some of the tools and devices, or may not which to have certain information monitored. Alternatively, the user may take the full recommendation at 3722, and purchase or otherwise obtain all recommended tools and devices. Thereafter, the user's activities are monitored and processed at 3724. The user is then presented with goal tracking reports at 3726.

The user may also be prompted to take baseline and periodic tests at 3728 to assess the severity of illness biomarkers or symptoms at start and over time. If the user chooses to take the test(s), the HIPS 100 can monitor the efficacy of the recommended activities in managing the illness, providing the user is meeting the recommended goals.

For example, in the context of Alzheimer's disease, a particular user may be provided with the following goals; 1) a certain number of hours of moderate exercise per week; 2) certain foods and supplements per day; 3) a certain numbers of hours of sleep per night; 4) certain hours of social activity; and 5) cognitive engagement. Goals 1, 3 and 5, for example, could be tracked by wearables and web portal tools, while goals 2 and 4 could be either manually tracked or tracked with the assistance of additional smart devices, e.g., smart refrigerator, smart pill packs, etc.

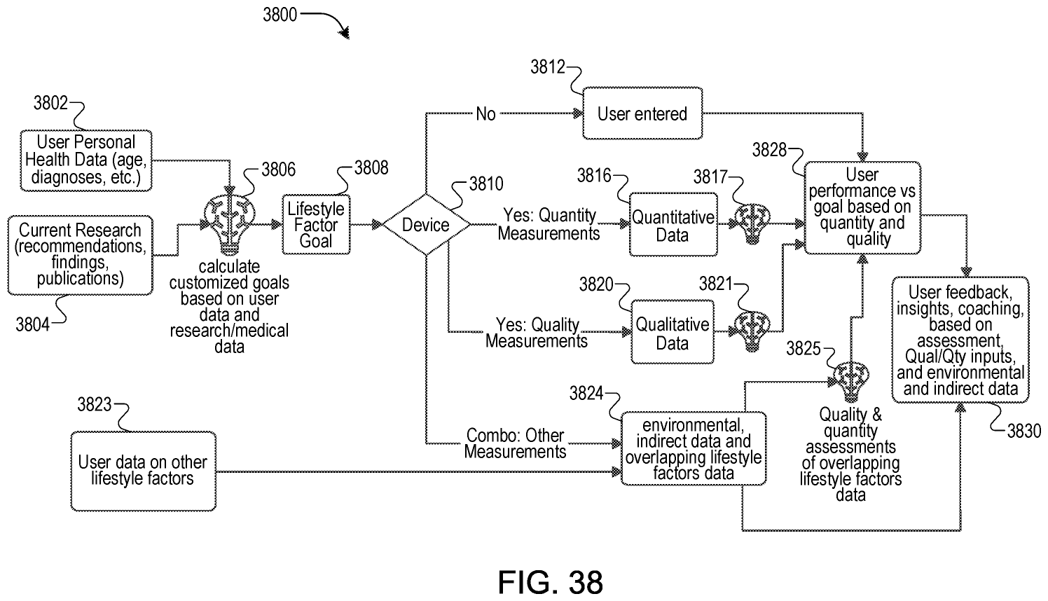

FIG. 38 is a system flow diagram 3800 of tracking lifestyle goals generated from the process of FIG. 37. User personal health data 3802 and data related to current research and goals are input to a goal processor 3806. The goal processor 3806 generates goals 3808 for the user. The process 3806 can be a rule based process or a machine learned process. For example, for a particular illness and a particular set of user factors, a particular set of predefined goals may be selected. Thus, for any illness, there may be multiple different sets of goals, where each set corresponds to a particular set of user factors.

Not all goals may be monitored by a tool or device. The HIPS 100 can determine at 3810 which goals will not be monitored by a tool or device, e.g., a user may need to apply sunscreen whenever outdoors, and thus the HIPS 100 will notify the user of user-required inputs and, optionally, generate an input tool 3812 for user entered input. Thus, when the HIPS 100 determines a data channel for monitoring a particular recommendation is not available, e.g., no tool or device is available, and that manual tracking must be used, the HIPS 100 proactively provides a monitoring input to the user that increases compliance. The illness metering system of the HIPS 100 thus identifies particular recommendations for which no automated data recording device is available, and in response generates data recordation queries to the user and delivers the queries to the user by electronic means. The queries include response options or fields that allow the user to quickly and easily provide data for recordation and goal tracking. Accordingly, data recordation and data integrity is increased relative to systems in which the user must initiate manual data recordation.

Goals may be measured quantitatively by quantitative data 3816 or qualitatively by qualitative data 3820. Examples of quantitative date may be blood pressure, glucose levels, etc., and can be measured by meters. Examples of qualitative data may be self-assessment response to questions issued to the user by use of a smart device, e.g., “On a scale of 1-5, how is your stress level today (1 is completely relaxed, 5 is very stressed). Please reply with a number from 1-5).” Other examples can be data characterizing the quantitative data, e.g., while a device may report that a user slept for eight hours, the device may also report that the user's sleep was of low quality, as indicated by frequent movement, heart rate, etc.

Other data 3824 can also be considered, such as device reported data, e.g., environmental (air quality and temperature at the user's current location, etc.), and user data on other lifestyle factors 3823, e.g., life changes (marriage, divorce, recent birth of a child, etc.), injuries (e.g., broken leg), and the like can also be considered.

The goal data are then processed at 3828 to assess user performance in meeting the goals, e.g., by meters though an application, a web portal, and the like. At 3830, user feedback can also be assessed. Such feedback may be, for example, the user rating the ability to meet each goal. For example, if a user has a physical injury, it may be difficult for the user to meet exercise goals for several weeks. Accordingly, showing goal meters in which one is at 0% may induce unnecessary stress, and thus the goal may be temporarily suspended until the user is able to resume exercises.

Thus, when HIPS 100 determines, based on health data of the user of the user device, for a particular goal of the set of goals, that the user cannot perform activities to progress in achieving the goal value, the HIPS 100 suspends the collection of data indicating progress of the user in achieving the particular goal value, and adjusts the determining of an overall goal achievement of the user by omitting data indicating progress of the user in achieving each goal value of the particular goal from the determination. When the HIPS 100 determines the user has recovered, then it resumes the collection of data indicating progress of the user in achieving the particular goal value, and adjusts determining of the overall goal achievement of the user by including the data indicating progress of the user in achieving each goal value of the particular goal from the determination.

Returning to the Alzheimer's disease example, the goals of sleep time, learning time, exercise time, diet intake, social interaction time, and stress management may be set for a user. Data indicating compliance with most of these goals can be in part or in whole captured passively through the user's devices and apps. The raw data is gathered and processed quantitatively and where applicable qualitatively, to provide simple feedback to the user in the form of a score.

The type of activity monitored may also be taken into account for goal compliance, with some activities contributing more for goal compliance than others do. For example, a 60 minute group yoga class might contribute 10 minutes to exercise (10 minutes of the 60 minutes the user achieved moderate or greater heart rate), 30 minutes to stress management (the qualitative component assigns 50% to the 60 minutes) and 10 minutes to social time (most class is individual but the qualitative component gives some credit to the minimal interactions during the class).

Activities such as learning can be facilitated by the HIPS 100. For example, articles and cognitive activities can be provided to the user via a device (e.g., a computer) that tracks user activity, such as eye tracking to measure if the user is reading the articles and user interactions to determine if the user is engaged with the cognitive activities. Based on the user's preferences, including disabilities, accessibilities (financial, physical, geographic, etc.), the HIPS 100 provides customized recommendations for new activities that suit the user's needs as well as feedback on the user's performance. Similar compliance tracking can be done for social goals and stress management.

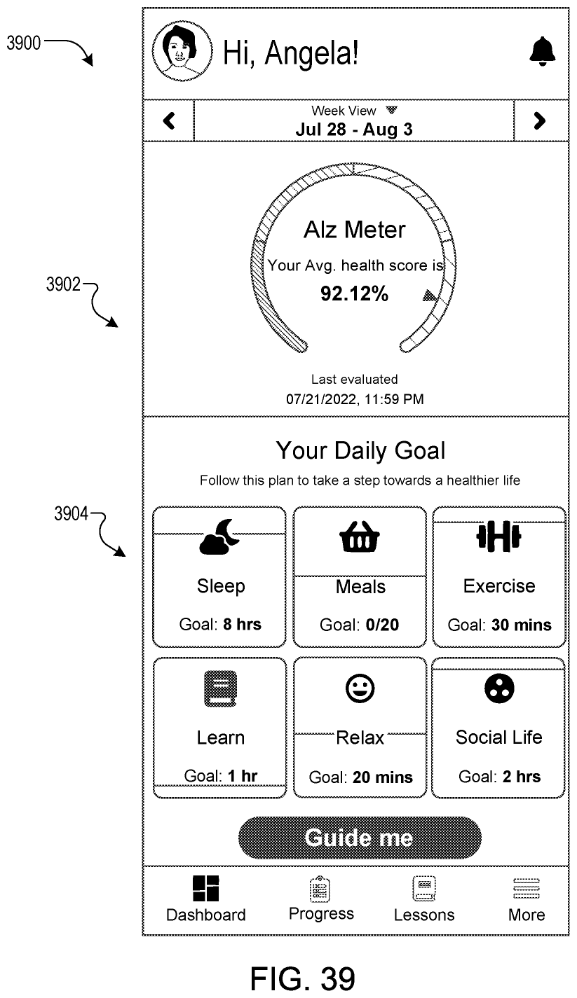

FIG. 39 is an illustration of a user interface 3900 of an illness metering application. The user interface includes an aggregate assessment score indicator 3902 that is based on compliance metrics that monitored for a set of recommendations 3904. For example, as shown in FIG. 39, the monitoring indicates the user has high compliance with sleep, exercise and social life recommendations, moderate compliance with the meals and relaxation recommendations, and low compliance with the learning requirement. However, the overall assessment is high, at 92%. This is because compliance with each recommendation may be weighted differently, and is dependent on the type of illness being monitored, particular health and physical factors of the individual, and how much each recommended activity/requirement contributes to management of the illness.

In some implementations, the illness metering and monitoring aspect includes determining, for a user device, a set of goals for tracking. The goals are specific to a particular condition, such as a first set of goals for Alzheimer's, a second, different set of goals for heart disease, and so on. The HIPs 100 then determines, based on health data of a user of a user device, a corresponding set of goal values for the set of goals. The goal values may be, for example, an amount of sleep for a sleep goal, and amount of exercise for an exercise goal, and so on. The values may differ between two people for a same set of goals, based on the particular health data of the two people.

The HIPS 100 receives, from one or more user devices of the user, data indicating progress of the user in achieving each goal value for each goal in the set of goals. Such data can include data describing an amount of sleep, data detailing exercise, etc. The HIPS 100 determines, from the data indicating progress of the user in achieving each goal value for each goal in the set of goals, an overall goal achievement of the user. Each goal can be weighted differently, and the overall goal achievement can be any appropriate function of the goal values. The HIPS 100 then provides, to the user device, data that causes the user device to display, for each goal in the set of goals, a progress measure in achieving the goal, and the overall goal achievement of the user. For example, data causing the display of FIG. 39 can be provided.

In some implementations, the HIPS 100 determines, for each goal of the set of goals for tracking, whether the goal is trackable by a user instrumentation. A goal can be tracked by instrumentation if it is determined that an activity can be sensed by a device and reported, e.g., footsteps, sleep, etc. Other goals, such as diet, stress levels, may require user recordation. For each goal that is determined to be trackable by a user instrumentation, the HIPS 100 determines whether the user account receives data from the user instrumentation for the goal. For example, if steps are to be counted and can be tracked by instrumentation, the HIPS 100 determines if the user has a wearable pedometer that can report the data. For each goal for which the user account does not receive data from the user instrumentation for the goal, the HIPS 100 generates data that causes a user device of the user to display a recommendation for one or more devices that perform the function of the user instrumentation. For example, the HIPS 100 may generate a recommendation for a particular pedometer and a link to a site to purchase the pedometer. For each goal that is determined to not be trackable by a user instrumentation, the HIPS 100 periodically provides data to a user device of the user that causes the user device to display an input user interface in which the user may enter the data indicating progress of the user in achieving the goal value. When the user inputs the data, the HIPS 100 receives, from the user device, the data indicating progress of the user in achieving the goal value.

In situations in which the systems discussed here collect personal information about users, or may make use of personal information, the users may be provided with an opportunity to control whether applications or features collect user information (e.g., information about a user's social network, social actions or activities, profession, a user's preferences, or a user's current location), or to control whether and/or how to receive content that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus.

A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., a FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, biometric assay, or tactile input. In addition, a computer can interact with a user by sending documents to and receiving documents from a device that is used by the user; for example, by sending web pages to a web browser on a user's user device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a user computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include users and servers. A user and server are generally remote from each other and typically interact through a communication network. The relationship of user and server arises by virtue of computer programs running on the respective computers and having a user-server relationship to each other. In some embodiments, a server transmits data (e.g., an HTML page) to a user device (e.g., for purposes of displaying data to and receiving user input from a user interacting with the user device). Data generated at the user device (e.g., a result of the user interaction) can be received from the user device at the server.

While this specification contains many specific implementation details, these should not be construed as limitations on the scope of any features or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

1. A system, comprising:
a data processing apparatus including a plurality of computers; and
a non-transitory computer readable memory storing instructions executable by the data processing apparatus and that upon such execution cause the data processing apparatus to perform operations comprising:
for each of a plurality of users:
establishing, for the user, a unique account for the user;
receiving, for the user, a set of data sources for the user, each data source being different from each other data source, and each data source requiring login credentials for the user;
for at least one or more of the data sources, establishing, by the system and without user input, login credentials for the user; and
storing in association with the unique account for the user, the login credentials for the user;
for the at least one or more data sources of the set of data sources for the user:
establishing, using the login credential for the data source established by the system, a connection to the data source;
receiving, from the data source, health data specific to the user; and
storing, in the unique account of the user, the health data for the user received from each data source in a data store.

2. The system of claim 1, the operations further comprising, for each unique account:
for data sources from which health data is received, determining whether the health data received from the data source requires data cleaning;
for a first data source for which health data is determined to not require data cleaning, storing the health data in the unique account of the user;
for a second data source for which health data is determined to require data cleaning:
determining whether changes resulting from the data cleaning trigger a user alert;
in response to determining the changes resulting from the data cleaning trigger the user alert:
notifying the user to validate the changes before persisting the changes; and
persisting the changes only in response to the user validating the change; and
in response to determining the changes resulting from the data cleaning do not trigger the user alert, persisting the changes.

3. The system of claim 1, the operations further comprising, for each unique account:
for each data source from which health data is received:
determining whether the health data received from the data source requires data cleaning, the determining comprising:
determining, for the health data received from the data source, a health data type;
determining, for the health data type, health data values required for the health data type;
determining whether the health data values in the health data received form the data source do not match the health data values required for the health data type;
for each data source for which the health data values of the health data received does not match the health data values required for the health data type, determining the health data received from the data source requires cleaning; and
for each data source for which the health data values of the health data received do match the health data values required for the health data type, determining the health data received from the data source does not require cleaning.

4. The system of claim 3, the operations further comprising, for health data from a data source determined to require cleaning:
determining health data values that are missing from the health data;
generating a query for the data source requesting the health data values that are determined to be missing from the health data; and
sending the query to the data source.

5. The system of claim 3, the operations further comprising, for health data from a data source determined to require cleaning:
determining health data values that are missing from the health data;
determining that at least one of the health data values is an index value that references the health data values that are missing from the health data in a health data database; and
querying the health data database using the index value to obtain the health data values that are missing from the health data; and
augmenting the health data with the health data values obtained from the health data.

6. The system of claim 1, wherein the operation of storing, in the unique account of the user, the health data for the user received from each data source, comprises:
for at least one data source, receiving health data in a non-standardized format, the non-standardized format being a format different from a standardized format in which health data is stored in the data store;
determining a conversion of the health data in the non-standardized format to the standardized format based on the non-standardized format;
converting, based on conversation, the health data from the non-standardized format to the standardized format; and
storing the converted health data in the standardized format in the data store.

7. The system of claim 1, the operations further comprising, for each unique account:
receiving, from the user of the account, share data specifying a plurality of share levels for the health data, wherein different portions of the health data are each associated with a different share level; and
for each portion of the health data associated with a particular share level, enabling sharing of the health data according to the share level.

8. The system of claim 7, the operations further comprising:
for a unique account of a user, receiving data describing a user obligation to share particular health data of the user with a third party;
determining that the share level of the particular health data precludes sharing of the particular health data with the third party;
in response to determining that the share level of the particular health data precludes sharing of the particular health data with the third party, determining a user preference for a share conflict; and
performing a share resolution process according to the user preference.

9. The system of claim 8, wherein:
determining a user preference for a share conflict comprises determining whether the user preference is an automatic override of the share level or a user sharing conformation;
in response to determining the user preference is an automatic override of the share level, allowing, without user confirmation, sharing of the particular health data with only the third party; and
in response to determining the user preference is user sharing confirmation, requesting the user confirm sharing of the particular health data with the third party.

10. The system of claim 1, the operations further comprising, for each unique account:
determining, based on the health data of the user, that a health metric value has deviated from a baseline value;
determining, based on the health metric value that has been determined to deviate from the baseline value, one or more questions to present to the user;
presenting the one or more questions to the user and storing the responses from the user to the one or more questions; and
based on the health metric values and the responses, determining potential causes that caused the health metric value to deviate from the baseline value.

11. The system of claim 1, the operations further comprising, for each unique account:
determining, based on the health data of the user, that a health metric value triggers a health inquiry;
determining, based on the health inquiry determination, one or more questions to present to the user;
presenting the one or more questions to the user and storing the responses from the user to the one or more questions; and
based on the health metric values and the responses, determining potential causes that caused the health metric value to trigger a health inquiry.

12. The system of claim 1, the operations further comprising, for each user:
determining, from the health data stored for the user account of the user, health preferences;
determining, from the health preferences, a set of type weights, wherein each type weight describes an estimated level of user interest in a particular heath service type;
selecting, based on the type weights, one or more health service recommendations for the user; and
providing, to the user, the one or more selected health service recommendations.

13. The system of claim 12, wherein the one or more health service recommendations include adjusting share levels of health data, a health product recommendation, a health action recommendation, and a health content stream recommendation.

14. The system of claim 1, the operations further comprising:
receiving, from one of the data sources, a health history request for a user, the health history request specifying a plurality of health data values of the user to be provided;
in response to the health history request, providing, to the one of the data sources, the health data values of the user.

15. The system of claim 14, wherein in response to the health history request, providing, to the one of the data sources, the health data values of the user, comprises:
sending, to the user, a request for confirmation to share the health data values to the one of the data sources; and
providing, in response to the health history request, to the one of the data sources, the health data values of the user only in response to receiving a confirmation from the user.

16. The system of claim 1, wherein the health data for one or more users include, for each of the one or more users, medications the user is taking and a schedule for each medication, and the operations further comprising, for each user of the one or more users:
determining a periodic dosing schedule for each medication, the dosing schedule indicating, for each medication, a time to administer the medication, the determining comprising:
for each medication, accessing medication data describing dosing requirements and constraints, medication interactions with other medications, and side effects of the medications;
determining, from the health data of the user, an activity pattern data of the user, the activity pattern data specifying user activities over multiple periodic dosing periods;
based on the medication data and the activity pattern data of the user, determining a periodic dosing schedule that specifies, for a time period and for each medication, a time to administer the medication;
sending, to a user device of a user for display to the user, the periodic dosing schedule; and
monitoring the dosing schedule for the user, the monitoring comprising at each time to administer a medication, sending, to the user device, a notification to the user to administer the medication.

17. The system of claim 16, wherein sending, to the user device, a notification to the user to administer the medication comprises sending, to an electronic pill package that stores the medications for one or more periodic dosing schedules, data that causes electronic pill package to generate an audible and/or visual notification for the user.

18. The system of claim 17, wherein the audible or visual notification specifies a specific compartment of the electronic pill package that stores a particular medication to be administered.

19. The system of claim 16, wherein based on the medication data and the activity pattern data of the user, determining a periodic dosing schedule that specifies, for a time period and for each medication, a time to administer the medication comprises determining the periodic dosing schedule for at least one medication based on a dependency of food ingestion.

20. The system of claim 17, the operations further comprising:
determining, based on the dosing schedule for a user, a quantity of medications to store in each of a plurality of pill receptacles in the electronic pill package; and
providing data that describes, to the user, the quantity of medications to store in each of a plurality of pill receptacles in the electronic pill package.

21. The system of claim 1, the operations further comprising:
determining, for a user account, a set of goals for tracking, wherein the goals are specific to a particular condition;
determining, based on health data of a user of the user account, a corresponding set of goal values for the set of goals;
receiving, from one or more user devices of the user, data indicating progress of the user in achieving each goal value for each goal in the set of goals;
determining, from the data indicating progress of the user in achieving each goal value for each goal in the set of goals, an overall goal achievement of the user; and
providing, to a user device associated with the user, data that causes the user device to display, for each goal in the set of goals, a progress measure in achieving the goal, and the overall goal achievement of the user.

22. The system of claim 21, the operations further comprising:
determining, for each goal of the set of goals for tracking, whether the goal is trackable by a user instrumentation;
for each goal that is determined to be trackable by a user instrumentation:
determining whether the user account receives data from the user instrumentation for the goal; and
for each goal for which the user account does not data from the user instrumentation for the goal, generating data that causes a user device of the user to display a recommendation for one or more devices that perform the function of the user instrumentation; and
for each goal that is determined to not be trackable by a user instrumentation, periodically providing data to a user device of the user that causes the user device to display an input user interface in which the user may enter the data indicating progress of the user in achieving the goal value, and receiving, from the user device, the data indicating progress of the user in achieving the goal value.

23. The system of claim 21, the operations further comprising:
determining, based on health data of the user of the user device, for a particular goal of the set of goals, that the user cannot perform activities to progress in achieving the goal value, and in response:
suspending the collection of data indicating progress of the user in achieving the particular goal value; and
adjusting the determining of the overall goal achievement of the user by omitting data indicating progress of the user in achieving each goal value of the particular goal from the determination.

24. The system of claim 23, the operations further comprising:
determining, based on health data of the user of the user device, for a particular goal for which it was determined that the user cannot perform activities to progress in achieving the goal value, that the user can perform activities to progress in achieving the goal value, and in response:
resuming the collection of data indicating progress of the user in achieving the particular goal value; and
adjusting the determining of the overall goal achievement of the user by including the data indicating progress of the user in achieving each goal value of the particular goal from the determination.

25. A non-transitory computer readable storage device storing instructions executable by one or more computers and that upon such execution cause the one or more computers to perform the operations of:
for each of a plurality of users:
establishing, for the user, a unique account for the user;
receiving, for the user, a set of data sources for the user, each data source being different from each other data source, and each data source requiring login credentials for the user;
for at least one or more of the data sources, establishing, by the system and without user input, login credentials for the user; and
storing in association with the unique account for the user, the login credentials for the user;
for the at least one or more data sources of the set of data sources for the user:
establishing, using the login credential for the data source established by the system, a connection to the data source;
receiving, from the data source, health data specific to the user; and
storing, in the unique account of the user, the health data for the user received from each data source in a data store.

26. A computer-implemented method comprising the steps of:
for each of a plurality of users:
establishing, for the user, a unique account for the user;
receiving, for the user, a set of data sources for the user, each data source being different from each other data source, and each data source requiring login credentials for the user;
for at least one or more of the data sources, establishing, by the system and without user input, login credentials for the user; and
storing in association with the unique account for the user, the login credentials for the user;
for the at least one or more data sources of the set of data sources for the user:
establishing, using the login credential for the data source established by the system, a connection to the data source;
receiving, from the data source, health data specific to the user; and
storing, in the unique account of the user, the health data for the user received from each data source in a data store.
